(19) United States
(12) Patent Application Publication
(10) Pub. No.: US 2022/0065785 A1
(43) Pub. Date: Mar. 3, 2022

(54) SYSTEM AND METHOD FOR DETECTING CHANGE POINTS

(71) Applicant: Quantum-Si Incorporated, Guilford, CT (US)

(72) Inventors: Minh Duc Cao, Belmont, MA (US); Mel Davey, Westbrook, CT (US)

(73) Assignee: Quantum-Si Incorporated, Guilford, CT (US)

(21) Appl. No.: 17/381,139

(22) Filed: Jul. 20, 2021

Related U.S. Application Data
(60) Provisional application No. 63/055,695, filed on Jul. 23, 2020.

(51) Int. Cl. G01N 21/64; G06F 17/18; G16B 30/10

(52) U.S. Cl. CPC G01N 21/6428; G06F 17/18; G01N 2021/6439; G16B 30/10; G01N 21/6408

(57) ABSTRACT

Techniques for evaluating change points in sequencing data during a sequencing reaction is described. The techniques may include obtaining data regarding light detected over time from luminescent labels associated with at least one molecule undergoing a sequencing reaction, evaluating candidate change points of the data within a time window of the data that varies over time, and outputting information identifying a set of change points based on evaluating the candidate change points. The set of change points may be used to determine pulse segments and individual nucleotides corresponding to at least some of the pulse segments.

### RELATED APPLICATION

This Application claims the benefit under 35 U.S.C. § 119(e) of U.S. Provisional Patent Application No. 63/055,695, filed Jul. 23, 2020, entitled “SYSTEM AND METHODS FOR DETECTING CHANGE POINTS ONLINE,” which is hereby incorporated by reference in its entirety.

### FIELD

Aspects of the present application relate to detection of change points in sequencing data during a sequencing reaction for one or more biological molecules (e.g., nucleic acid, peptide). The detected change points may be used for identifying a series of subunits (e.g., nucleotides, amino acids) of a biological molecule.

### BACKGROUND

Sequencing of nucleic acids (e.g., deoxyribonucleic acid (DNA), ribonucleic acid (RNA)) includes identifying individual nucleotides in a target nucleic acid. Some nucleic acid sequencing methods include identifying individual nucleotides as they are incorporated into a nucleic acid strand complementary to the target nucleic acid being sequenced. The series of nucleotides for the complementary strand identified during the sequencing process may then allow for the identification of the nucleotide sequence for the target nucleic acid strand.

### SUMMARY

According to an aspect of the present application, a method for detecting change points online is provided. The method comprises obtaining data regarding light detected over time from luminescent labels associated with at least one molecule undergoing a sequencing reaction; evaluating candidate change points of the data within a time window of the data that varies over time; and outputting information identifying a set of change points based on evaluating the candidate change points.

According to an aspect of the present application, a system comprising at least one hardware processor and at least one non-transitory computer-readable storage medium storing processor executable instructions that, when executed by the at least one hardware processor, cause the at least one hardware processor to perform a method. The method comprises obtaining data regarding light detected over time from luminescent labels associated with at least one molecule undergoing a sequencing reaction; evaluating candidate change points of the data within a time window of the data that varies over time; and outputting information identifying a set of change points based on evaluating the candidate change points.

According to an aspect of the present application, at least one non-transitory computer-readable medium storing processor-executable instructions that, when executed by at least one hardware processor, cause the at least one hardware processor to perform a method. The method comprises obtaining data regarding light detected over time from luminescent labels associated with at least one molecule undergoing a sequencing reaction; evaluating candidate change points of the data within a time window of the data that varies over time; and outputting information identifying a set of change points based on evaluating the candidate change points.

According to an aspect of the present application, a sequencing instrument comprising at least one photodetector configured to receive light from luminescent labels during a sequencing reaction, at least one hardware processor, and at least one non-transitory computer-readable storage medium storing processor-executable instructions that, when executed by the at least one hardware processor, cause the at least one hardware processor to perform a method. The method comprises obtaining data regarding light detected over time from luminescent labels associated with at least one molecule undergoing a sequencing reaction; evaluating candidate change points of the data within a time window of the data that varies over time; and outputting information identifying a set of change points based on evaluating the candidate change points.

According to an aspect of the present application, a method for detecting change points online is provided. The method comprises obtaining data regarding light detected over time from luminescent labels associated with at least one molecule undergoing a sequencing reaction; and detecting change points of the data during the sequencing reaction.

According to an aspect of the present application, a system comprising at least one hardware processor and at least one non-transitory computer-readable storage medium storing processor executable instructions that, when executed by the at least one hardware processor, cause the at least one hardware processor to perform a method. The method comprises obtaining data regarding light detected over time from luminescent labels associated with at least one molecule undergoing a sequencing reaction; and detecting change points of the data during the sequencing reaction.

According to an aspect of the present application, at least one non-transitory computer-readable medium storing processor-executable instructions that, when executed by at least one hardware processor, cause the at least one hardware processor to perform a method. The method comprises obtaining data regarding light detected over time from luminescent labels associated with at least one molecule undergoing a sequencing reaction; and detecting change points of the data during the sequencing reaction.

According to an aspect of the present application, a sequencing instrument comprising at least one photodetector configured to receive light from luminescent labels during a sequencing reaction, at least one hardware processor, and at least one non-transitory computer-readable storage medium storing processor-executable instructions that, when executed by the at least one hardware processor, cause the at least one hardware processor to perform a method. The method comprises obtaining data regarding light detected over time from luminescent labels associated with at least one molecule undergoing a sequencing reaction; and detecting change points of the data during the sequencing reaction.

According to an aspect of the present application, a method for detecting change points online is provided. The method comprises obtaining first data regarding light detected from at least one luminescent label associated with at least one molecule undergoing a sequencing reaction; categorizing at least one first candidate change point of the first data as not being a change point; after the categorizing, obtaining second data regarding light detected from at least one luminescent label associated with the at least one molecule undergoing the sequencing reaction; and evaluating at least one second candidate change point based at least in part on the second data.

According to an aspect of the present application, a system comprising at least one hardware processor and at least one non-transitory computer-readable storage medium storing processor executable instructions that, when executed by the at least one hardware processor, cause the at least one hardware processor to perform a method. The method comprises obtaining first data regarding light detected from at least one luminescent label associated with at least one molecule undergoing a sequencing reaction; categorizing at least one first candidate change point of the first data as not being a change point; after the categorizing, obtaining second data regarding light detected from at least one luminescent label associated with the at least one molecule undergoing the sequencing reaction; and evaluating at least one second candidate change point based at least in part on the second data.

According to an aspect of the present application, at least one non-transitory computer-readable medium storing processor-executable instructions that, when executed by at least one hardware processor, cause the at least one hardware processor to perform a method. The method comprises obtaining first data regarding light detected from at least one luminescent label associated with at least one molecule undergoing a sequencing reaction; categorizing at least one first candidate change point of the first data as not being a change point; after the categorizing, obtaining second data regarding light detected from at least one luminescent label associated with the at least one molecule undergoing the sequencing reaction; and evaluating at least one second candidate change point based at least in part on the second data.

According to an aspect of the present application, a sequencing instrument comprising at least one photodetector configured to receive light from luminescent labels during a sequencing reaction, at least one hardware processor, and at least one non-transitory computer-readable storage medium storing processor-executable instructions that, when executed by the at least one hardware processor, cause the at least one hardware processor to perform a method. The method comprises obtaining first data regarding light detected from at least one luminescent label associated with at least one molecule undergoing a sequencing reaction; categorizing at least one first candidate change point of the first data as not being a change point; after the categorizing, obtaining second data regarding light detected from at least one luminescent label associated with the at least one molecule undergoing the sequencing reaction; and evaluating at least one second candidate change point based at least in part on the second data.

According to an aspect of the present application, a method for detecting change points online is provided. The method comprises obtaining first data regarding light detected over time from luminescent labels associated with at least one molecule undergoing a sequencing reaction; evaluating candidate change points of the first data; obtaining second data regarding light detected from at least one luminescent label associated with the at least one molecule undergoing the sequencing reaction; and re-evaluating the candidate change points based at least in part on the second data.

According to an aspect of the present application, a system comprising at least one hardware processor and at least one non-transitory computer-readable storage medium storing processor executable instructions that, when executed by the at least one hardware processor, cause the at least one hardware processor to perform a method. The method comprises obtaining first data regarding light detected over time from luminescent labels associated with at least one molecule undergoing a sequencing reaction; evaluating candidate change points of the first data; obtaining second data regarding light detected from at least one luminescent label associated with the at least one molecule undergoing the sequencing reaction; and re-evaluating the candidate change points based at least in part on the second data.

According to an aspect of the present application, at least one non-transitory computer-readable medium storing processor-executable instructions that, when executed by at least one hardware processor, cause the at least one hardware processor to perform a method. The method comprises obtaining first data regarding light detected over time from luminescent labels associated with at least one molecule undergoing a sequencing reaction; evaluating candidate change points of the first data; obtaining second data regarding light detected from at least one luminescent label associated with the at least one molecule undergoing the sequencing reaction; and re-evaluating the candidate change points based at least in part on the second data.

According to an aspect of the present application, a sequencing instrument comprising at least one photodetector configured to receive light from luminescent labels during a sequencing reaction, at least one hardware processor, and at least one non-transitory computer-readable storage medium storing processor-executable instructions that, when executed by the at least one hardware processor, cause the at least one hardware processor to perform a method. The method comprises obtaining first data regarding light detected over time from luminescent labels associated with at least one molecule undergoing a sequencing reaction; evaluating candidate change points of the first data; obtaining second data regarding light detected from at least one luminescent label associated with the at least one molecule undergoing the sequencing reaction; and re-evaluating the candidate change points based at least in part on the second data.

According to an aspect of the present application, a method for detecting change points online is provided. The method comprises obtaining data regarding light detected over time from luminescent labels associated with at least one molecule undergoing a sequencing reaction; evaluating a first candidate change point of the data as being a change point; evaluating, for a portion of the data occurring at a time after the first candidate change point, a second candidate change point as being a change point; and updating a set of change points to include the second candidate change point.

According to an aspect of the present application, a system comprising at least one hardware processor and at least one non-transitory computer-readable storage medium storing processor executable instructions that, when executed by the at least one hardware processor, cause the at least one hardware processor to perform a method. The method comprises obtaining data regarding light detected over time from luminescent labels associated with at least one molecule undergoing a sequencing reaction; evaluating a first candidate change point of the data as being a change point; evaluating, for a portion of the data occurring at a time after the first candidate change point, a second candidate change point as being a change point; and updating a set of change points to include the second candidate change point.

According to an aspect of the present application, at least one non-transitory computer-readable medium storing processor-executable instructions that, when executed by at least one hardware processor, cause the at least one hardware processor to perform a method. The method comprises obtaining data regarding light detected over time from luminescent labels associated with at least one molecule undergoing a sequencing reaction; evaluating a first candidate change point of the data as being a change point; evaluating, for a portion of the data occurring at a time after the first candidate change point, a second candidate change point as being a change point; and updating a set of change points to include the second candidate change point.

According to an aspect of the present application, a sequencing instrument comprising at least one photodetector configured to receive light from luminescent labels during a sequencing reaction, at least one hardware processor, and at least one non-transitory computer-readable storage medium storing processor-executable instructions that, when executed by the at least one hardware processor, cause the at least one hardware processor to perform a method. The method comprises obtaining data regarding light detected over time from luminescent labels associated with at least one molecule undergoing a sequencing reaction; evaluating a first candidate change point of the data as being a change point; evaluating, for a portion of the data occurring at a time after the first candidate change point, a second candidate change point as being a change point; and updating a set of change points to include the second candidate change point.

### BRIEF DESCRIPTION OF DRAWINGS

Various aspects and embodiments will be described with reference to the following figures. The figures are not necessarily drawn to scale.

FIG. 1 is a diagram of an illustrative processing pipeline 100 for detecting change points in sequencing data and generating output sequences, in accordance with some embodiments of the technology described herein.

FIG. 2 is a flow chart of an illustrative process 200 for obtaining data, evaluating candidate change points, pruning prior candidate change points, and determining pulse segments, in accordance with some embodiments of the technology described herein.

FIG. 3A is an exemplary trace of sequencing data, in accordance with some embodiments of the technology described herein.

FIG. 3B is a schematic of detecting change points in sequencing data and generating an output sequence, in accordance with some embodiments of the technology described herein.

FIG. 4 is a flow chart of an illustrative process for evaluating candidate change points, in accordance with some embodiments of the technology described herein.

FIG. 5 is a flow chart of an illustrative process for detecting change points during a sequencing reaction, in accordance with some embodiments of the technology described herein.

FIG. 6 is a flow chart of an illustrative process for evaluating candidate change points, in accordance with some embodiments of the technology described herein.

FIG. 7 is a flow chart of an illustrative process for evaluating candidate change points, in accordance with some embodiments of the technology described herein.

FIG. 8 is a flow chart of an illustrative process for evaluating candidate change points, in accordance with some embodiments of the technology described herein.

FIG. 9 is a block diagram of an illustrative computer system that may be used in implementing some embodiments of the technology described herein.

### DETAILED DESCRIPTION

Computational techniques described herein relate to the sequencing of nucleic acids, such as DNA and RNA, and in particular to techniques for streaming the pulse calling process of the sequencing data during the sequencing reaction. Nucleic acid sequencing allows for the determination of the order and position of nucleotides in a target nucleic acid. Some nucleic acid sequencing methods are based on sequencing by synthesis, in which the identity of a nucleotide is determined as the nucleotide is incorporated into a newly synthesized strand of nucleic acid that is complementary to the target nucleic acid. During sequencing, a polymerizing enzyme (e.g., DNA polymerase) may couple (e.g., attach) to a priming location of a target nucleic acid molecule and add or incorporate nucleotides to the primer via the action of the polymerizing enzyme, which can be generally referred to as a primer extension reaction.

Conventional computational techniques for analyzing such sequencing data generally involves analyzing the entire data trace after the sequencing reaction has been performed. The analysis involves determining locations within the data trace as being change points that signal regions of the data corresponding to nucleotide incorporation events where there may have a relatively higher intensity level in comparison to other regions of the data. Those regions may be referred to as “pulses” within the sequencing data. Identifying specific nucleotides associated with those pulses may involve analyzing one or more features of the data within those regions to identify which base corresponds to a particular pulse. This process may be referred to as “base calling.”

The typical computational processing capabilities needed to perform pulse calling and base calling on an entire trace of sequencing data are significant because of the high amount of data included in an entire sequencing data trace. Conventional nucleic acid sequencing instruments do not generally have the computational processing capability to both collect the sequencing data and perform subsequent pulse and base calling on the data. Instead, once the sequencing data is obtained it is generally transferred to an external computational device (e.g., server) which is used to perform subsequent analysis.

The inventors have recognized and appreciated that these conventional techniques are limited because a user (e.g., researcher, physician) is only able to view the quality of the sequencing data after the sequencing reaction is complete and the data has been analyzed. Sequencing reactions can take significant amounts of time, particularly as longer sequence reads is achieved, and the inability for the user to monitor the quality of the sequencing reaction as it is occurring, and stop the reaction if the quality is not at a desired level, is a significant limitation in many conventional sequencing analysis pipelines.

The inventors have developed new computational techniques for analyzing sequencing data that allows for change points within the data, and pulses, to be detected on portions of the data such that the analysis of the data can run concurrently with the sequencing reaction. Such techniques allow for real-time monitoring of the sequencing reaction as additional sequencing data is obtained. According to the techniques of the present application, a change point may be estimated as being a data point where the signal within the data statistically changes with respect to the surrounding data points.

In addition, these techniques developed by the inventors are performed on portions of the sequencing data and require significantly less computational processing capabilities than if the analysis was performed on sequencing data for an entire sequencing reaction. Accordingly, some embodiments of the present application allow for detecting change points in real-time by a sequencing instrument, which may be referred to as detecting change points online. In such embodiments, the sequencing instrument may detect individual change points and determine regions of the sequencing data associated with pulses. An external computational device (e.g., server, cloud computing system) may receive data associated with those regions and perform a base calling process to determine nucleotides for individual pulses. In some instances, the sequencing instrument may detect a pulse and transmit data associated with the pulse to the external computational device. This may occur each time a pulse is detected. In some embodiments, the sequencing instrument may not only detect the individual change points but also perform pulse calling, such as by using the change points to determine regions of the sequencing data associated with pulses, and base calling. In such instances, the sequencing instrument itself may output one or more nucleotide sequences.

These techniques provide certain benefits in reducing the amount of computational storage needed when processing the sequencing data using conventional techniques, which typically involve the use of a multi-computer server or cloud computing system. This is particularly the case where hundreds of thousands to a million sequencing reactions are occurring simultaneously and data from these sequencing reactions is being processed in real-time. For example, a real-time sequencing reaction typically produces approximately 50 events (e.g., nucleotide incorporation events) per second and data buffering is needed to handle the massive amount of data being obtained when hundreds of thousands to a million sequencing reactions are occurring. The change point detection techniques described herein allow for detection of change points using portions of the sequencing data, which allow for the change point detection process to occur during the sequencing reactions and reduce the need for buffering of the sequencing data. In some instances, the techniques described herein may be used where there is a fixed amount of memory. For example, these techniques may allow for a memory having the capacity to store approximately 12 data points*2 time bins*2 bytes per a sample well.

Although the technology described herein is generally described in the context of nucleic acid sequencing, it should be appreciated that these techniques may be used for analyzing other types of sequencing data, such as peptide sequencing data. In the context of peptide sequencing, the pulses may correspond to association events corresponding to detecting individual amino acids of a peptide.

Some embodiments described herein address all of the above-described issues that the inventors have recognized with evaluating change points in sequencing data online. However, not every embodiment described herein addresses every one of these issues, and some embodiments may not address any of them. As such, it should be appreciated that embodiments of the technology described herein are not limited to addressing all or any of the above-discussed issues with evaluating change points in sequencing data online.

Some embodiments involve obtaining data regarding light detected over time from luminescent labels associated with one or more molecules (e.g., DNA, RNA) undergoing a sequencing reaction (e.g., primer extension reaction). Candidate change points of the data may be evaluated within a time window of the data that varies over time, which may be considered as a sliding time window. Initially, all data points may be evaluated as being candidate change points, and as additional data is obtained, individual data points may be categorized as being or not being a change point. Based on evaluating the candidate change points within the time window that varies over time, a set of change points within the data is obtained.

In some embodiments, obtaining the sequencing data further comprises receiving portions of the data at different times. In such embodiments, after receiving one or more portions of the data, the time window of the data may be adjusted to include the one or more portions of data. In some embodiments, evaluating candidate change points involves evaluating candidate change points of the data within a first time window of the data, generating a second time window at least in part by adding, to the first time window, one or more data points occurring after the first time window, and evaluating candidate change points of the data within the second time window.

In some embodiments, after a change point of the data is estimated and subsequent evaluation of candidate change points may involve analyzing data occurring after the estimated change point. In such embodiments, any candidate change points occurring prior to the estimated change point may be removed from consideration as possible candidate change points. In some instances, candidate change points being evaluated after the change point may involve using at least some of the data occurring prior to the estimated change point. Such techniques allow for both pruning of candidate change points after a change point has been detected, but also allows for change points to be detected as a high level of accuracy by using data occurring prior to detected change points.

According to some embodiments of the present application, evaluating the candidate change points is performed at least in part by using one or more statistical models that estimate the likelihood of the candidate change points being change points. In some instances, one or more statistical models may be applied to the time window to estimate at least one change point.

In some embodiments, the change points may be used to determine segments of the data as being background signal or pulse segments. The pulse segments may correspond to nucleotide incorporation events. Segments of the data may be determined as being background signal or pulse segments by comparing values of one or more features of the data (e.g., intensity signal, signal variance) within the segment to a threshold value. If the values of the one or more features of the data are above the threshold value, then a particular segment may be identified as being a pulse segment. If the values of the one or more features of the data are below the threshold value, then a particular segment may be identified as being a background signal. In some embodiments, multiple features may be used to identify a particular segment as being a pulse segment or a background signal. In such embodiments, the multiple features may be combined to compute one or more measures, which can then be used to compare to a threshold value(s).

It should be appreciated that the various aspects and embodiments described herein be used individually, all together, or in any combination of two or more, as the technology described herein is not limited in this respect.

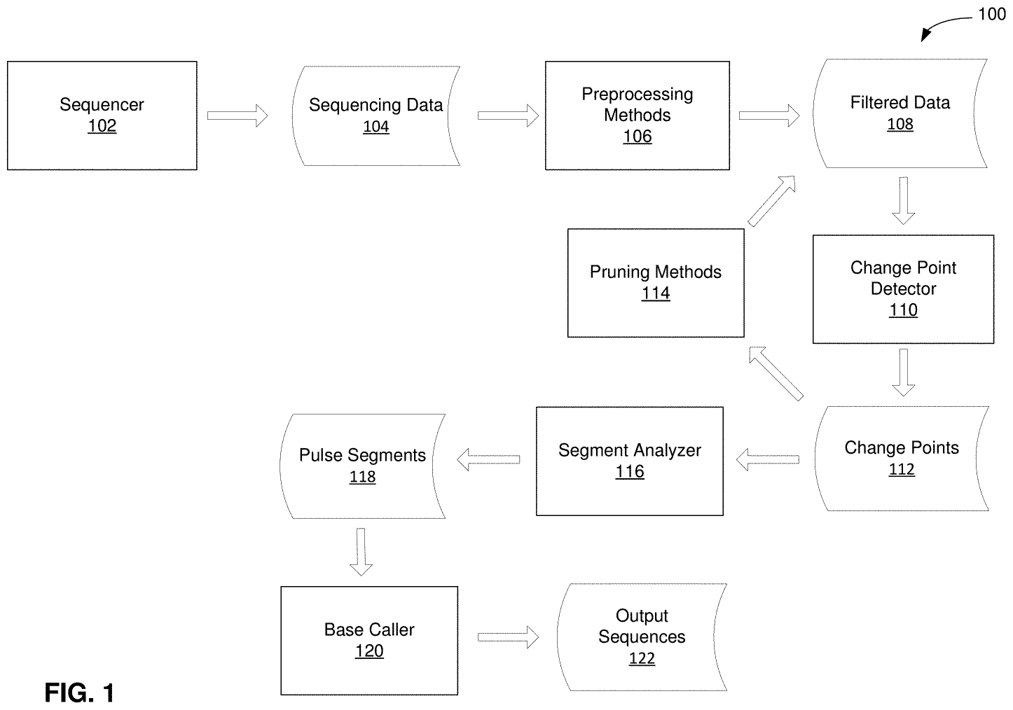

FIG. 1 is a diagram of an illustrative processing pipeline 100 for detecting change points in sequencing data and generating output sequences, which may be performed during the sequencing reaction, in accordance with some embodiments of the technology described herein.

As shown in FIG. 1, pipeline 100 includes sequencer 102 configured to generate sequencing data 104. In some embodiments, sequencer 102 is configured to sequence a nucleic acid sample and generate nucleic acid sequencing data for the sample.

Sequencing data 104 may be indicative of light detected over time from luminescent labels (e.g., fluorophores) associated with one or more molecules undergoing a sequencing reaction. In some embodiments, sequencer 104 may obtain sequencing data 104 during the sequencing reaction. In such instances, the sequencing data 104 may be considered to be obtained in “real-time” as the sequencing reaction occurs. According to some embodiments, sequencing data 104 may identify different characteristics of the detected light, including an intensity characteristic of the light and a temporal characteristic of the light. In some embodiments, sequencer 102 may have one or more detectors configured to detect light during one or more time bins, and a temporal characteristic of the light may be a ratio of photons detected in different time bins. In some embodiments, obtaining sequencing data 104 may involve obtaining time-bin information regarding the times at which the luminescent labels emit light in response to excitations of the luminescent labels. Obtaining sequencing data 104 may include calculating light intensity information based on the time-bin information, which may involve summing the time-bin information for individual times of the data. Examples of detectors that may be used in obtaining sequencing data are described in U.S. Patent Publication No. 2018/0180456, titled “INTEGRATED PHOTODETECTOR WITH DIRECT BINNING PIXEL,” filed Dec. 22, 2017, which is incorporated herein by reference in its entirety. Additional examples of detectors that may be used in obtaining sequencing data are described in U.S. Pat. No. 9,759,658, tided “INTEGRATED DEVICE FOR TEMPORAL BINNING OF RECEIVED PHOTONS,” filed Aug. 7, 2015, which is incorporated herein by reference in its entirety.

In should be appreciated that the techniques described herein may be used on different types of sequencing data. In some embodiments, the sequencing reaction is a synthesis reaction (e.g., sequencing by synthesis reaction performed detecting nucleotide incorporation events as part of real-time DNA sequencing). For example, in some embodiments, the sequencing reaction is a primer extension reaction. In such embodiments, sequencing data 104 may be indicative of light detected from luminescent labels during one or more nucleotide incorporation events. In some embodiments, the sequencing reaction is a degradation reaction (e.g., protein degradation reaction performed as part of detecting individual amino acids that form a protein sequence).

The luminescent labels may be attached to the one or more molecules using any suitable type of linker. In some embodiments, the luminescent labels correspond to nucleotides being incorporated into a nucleotide sequence during the sequencing reaction. In some embodiments, the luminescent labels correspond to amino acids of a peptide.

Some embodiments may involve preprocessing of the sequencing data prior to evaluating candidate change points in the sequencing data. As shown in FIG. 1, preprocessing methods 106 may be performed on sequencing data to generate filtered sequencing data 108. Preprocessing methods 106 may involve smoothing the raw sequencing data, which may improve subsequent change point detection performed by change point detector 110. For example, preprocessing methods 106 may involve removing or reducing outlier data points that may originate from noise in sequencing data 104 which may otherwise be identified as a candidate change point, and possibly further evaluated as a change point. In some embodiments, preprocessing methods may reduce noise introduced by laser drift occurring while the sequencing data is obtained.

According to aspects of the present application, change point detector 110 may be used to estimate change points 112 occurring in filtered sequencing data 108. Estimating change points 112 by change point detector 110 may involve estimating a change point of the data and evaluating candidate change points of the data occurring after the estimated change point. Change point detector 110 may involve estimating a first change point of the data by evaluating a first set of candidate change points, and estimating a second change point of the data by evaluating a second set of candidate change points occurring after the first change point. As change point detector 110 estimates additional change points, a set of change points 112 may be updated to include the additional change points.

In some embodiments, data occurring prior to the change point may be used in evaluating candidate change points of the data occurring after the estimated change point. For example, estimating a first change point may involve evaluating a first set of candidate change points using first data and estimating a second change point may involve evaluating a second set of candidate change points using second data that includes one or more data points of the first data. The second set of candidate change points may include one or more candidate change points of the first set occurring after the first change point. Such techniques may improve the accuracy of estimating individual change points while reducing the amount of data being evaluated by change point detector 110 at any given time.

In some embodiments, change point detector 110 may evaluate candidate change points of the data within a time window of the data that varies over time, which may be referred to as a sliding time window. Such techniques may involve change point detector 110 obtaining data by receiving different portions of the data at different times. After one portion of the data is received by change point detector 110, the time window of the data may be adjusted to include one or more portions of the data. In some embodiments, change point detector 110 may involve evaluating candidate change points of the data within a first time window of the data and generating a second time window by adding one or more data points occurring after the first time window to the first time window. In turn, the time window effectively has extended over a different duration of time to include additional data points in the sequencing data. Additional candidate change points may be then evaluated within the second time window.

Change point detector 110 may involve using one or more statistical models that estimate the likelihood of a particular candidate change point being a change point within the sequencing data. Evaluating individual candidate change points may involve applying the one or more statistical models to the time window to estimate one or more change points within the time window. The one or more statistical models may be used to calculate one or more scores for a candidate change point. The one or more scores may be then used to determine whether the candidate change point is a change point. In some instances, if the score is above a threshold value, then the candidate change point is a change point, and if the score is below the threshold value, then the candidate change point is not a change point. According to some embodiments, initially, all data points within a time window may be determined to be candidate change points and the one or more statistical models may then be used to evaluate all the data points, such as by calculating scores for the data points.

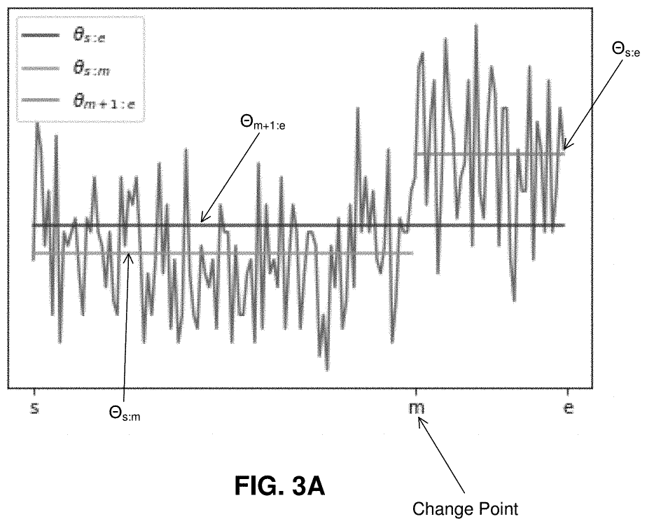

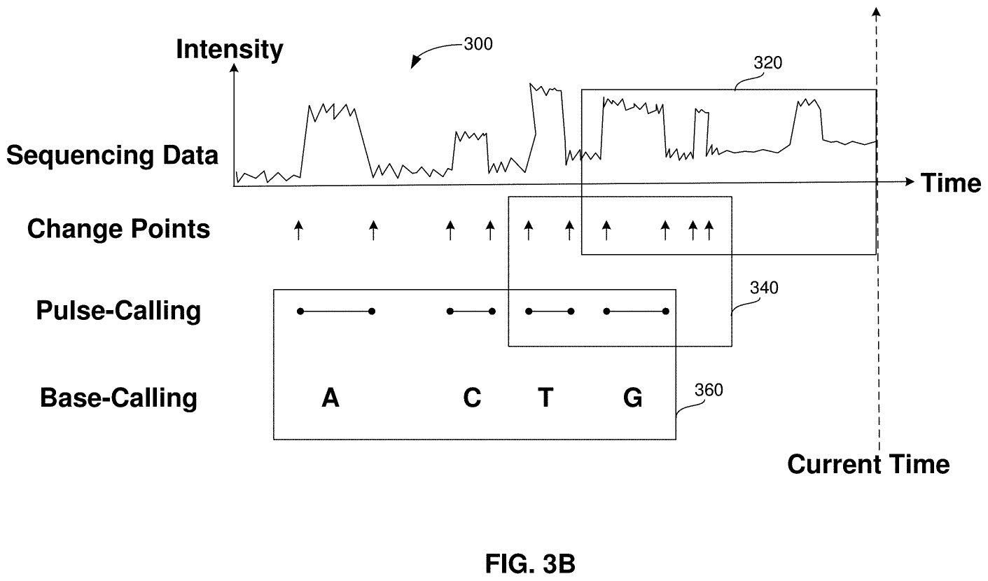

In some embodiments, change point detector 110 may categorize a candidate change point by calculating one or more values of a parameter associated with the candidate change point and using the one or more values to determine that the candidate change point is or is not a change point. In such embodiments, the candidate change point may be categorized by comparing the one or more values to a threshold value. For example, if a value of the parameter associated with a particular candidate change point is above a threshold value, then the candidate change point may be determined to be a change point. Similarly, if the value of the parameter is below the threshold value, then the candidate change point may be determined to not be a change point. Additional discussion of statistical models and their parameters that may be used in evaluating candidate change points is described herein including, for example, with respect to FIG. 3A.

Techniques for estimating change points 112 may involve using pruning methods 114, which may include removing one or more candidate change points occurring in the data prior to an estimated change point detected by change point detector 110. In some embodiments, after a first change point is estimated by change point detector 110, pruning methods 114 may update candidate change points by removing one or more candidate change points occurring in the sequencing data at a time prior to the first change point. The updated candidate change points may then be re-evaluated by change point detector 110, and in some embodiments, one or more additional change points may be estimated.

Some embodiments of the present application relate to obtaining additional sequencing data after determining that a candidate change point is not a change point. In such embodiments, sequencer 102 may obtain first data and change point detector 110 may categorize one or more candidate change points of the first data as not being a change point, such as by using one or more statistical models described herein. Then, sequencer 102 may obtain second data and change point detector 110 may evaluate one or more candidate change points based on the second data. In some embodiments, categorizing the one or more candidate change points of the first data may involve determining that a change point does not exist within the first data. Sequencer 102 may obtain second data, and change point detector 110 may re-evaluate the candidate change points based on the second data. In some embodiments, re-evaluating the candidate change points may be based on a portion of the first data and the second data. In some instances, evaluating candidate change points of the first data may involve using one or more statistical models to calculate scores for the candidate change points based on the first data, such as by comparing the scores to a threshold value. Re-evaluating the candidate change points may involve using the one or more statistical models to calculate scores for the candidate change points based on the first data and the second data.

According to some embodiments, change points 112 may be used to determine one or more nucleotide incorporation events. Segment analyzer 116 may involve determining segments of the data between individual change points of the set of change points as being background signal or as pulses, which may correspond to nucleotide incorporation events. Segment analyzer may output the portions of the data corresponding to the pulses as pulse segments 118.

In some embodiments, segment analyzer 116 involves determining segments of the data between individual change points as corresponding to background signal by comparing values of one or more features (e.g., signal intensity) of the data within the segments to a threshold value. Segments of the data having values of the one or more features below a threshold value may be determined as corresponding to a background signal. Segments of the data may be determined as being nucleotide incorporation events by comparing values of the one or more features of the data within the segments to the threshold value and identifying segments having values of the one or more features above the threshold value as corresponding to nucleotide incorporation events.

Pulse segments 118 may be passed to base caller 120, which in turn generates output sequences 122. In the context of nucleic acid sequencing, pulse segments 118 correspond to nucleotide incorporation events and base caller 120 may assign individual pulse segments to different types of nucleotides, which may be used to generate output sequences 122. One or more features of the data within a pulse segment (e.g., intensity, temporal information) may be used to assign a type of nucleotide (e.g., A, T, G, C) to the pulse segment. For example, a first pulse segment may include data indicating that an “A” type nucleotide as being incorporated into a growing nucleic acid strand during the time within the pulse segment. Assigning a type of nucleotide for the first pulse segment may include assigning an “A” type nucleotide to the first pulse segment. Similarly, a second pulse segment, occurring in time after the first pulse segment, may include data indicating that a “G” type nucleotide as being incorporated into the growing strand. Subsequently, a third pulse segment, occurring in time after the second pulse segment, may include data indicating that a “T” type nucleotide as being incorporated in the growing strand. With this assignment of nucleotides to the different pulse segments, the nucleotide sequence may be identified as being “AGT.” In some embodiments, base caller 120 may involve using clustering techniques to identify groups of pulse segments as being different types of nucleotides. Examples of base calling techniques that may be performed by base caller 120 are described in U.S. Patent Publication No. 2019/0237160, titled “MACHINE LEARNING ENABLED PULSE AND BASE CALLING FOR SEQUENCING DEVICES,” filed Jan. 25, 2019, which is incorporated herein by reference in its entirety. Additional base calling techniques that may be performed by base caller 120 are described in U.S. Patent Publication No. 2017/0349944, titled “PULSE CALLER AND BASE CALLER,” filed Jun. 1, 2017, which is incorporated herein by reference in its entirety.

In some embodiments, a sequencing instrument may include some or all of sequencer 102, preprocessing methods 106, change point detector 110, pruning methods 114, segment analyzer 116, and base caller 120.

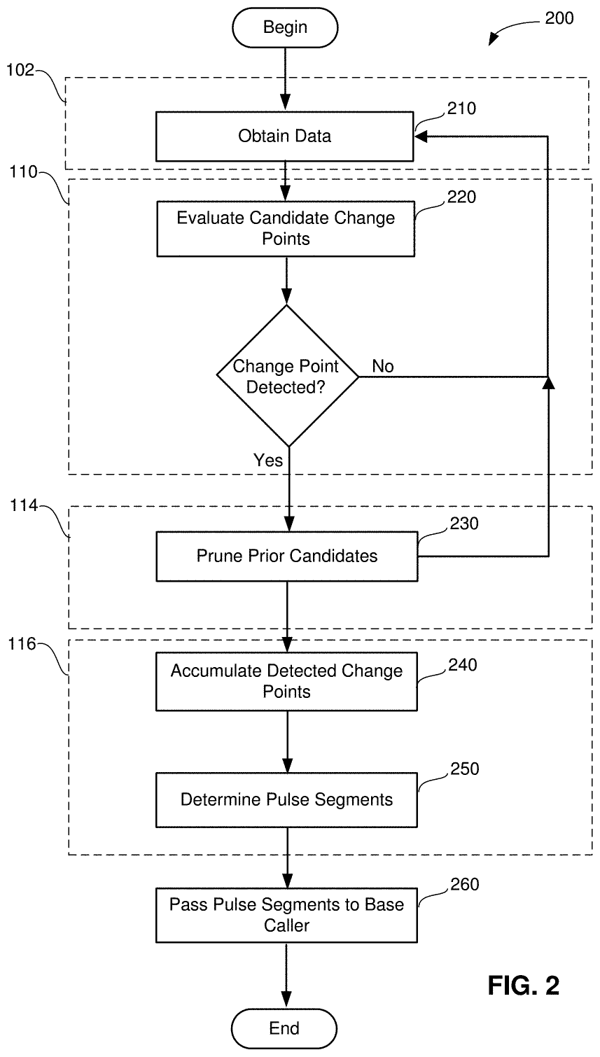

FIG. 2 is a flow chart of an illustrative process 200 for obtaining data, evaluating candidate change points, pruning prior candidate change points, and determining pulse segments, in accordance with some embodiments of the technology described herein. Process 200 may be performed on any suitable computing device(s) (e.g., a single computing device, multiple computing devices co-located in a single physical location or located in multiple physical locations remote from one another, one or more computing devices part of a cloud computing system, etc.), as aspects of the technology described herein are not limited in this respect. In some embodiments, sequencer 102, change point detector 110, pruning methods 114, segment analyzer 116, may perform some or all of process 200 to evaluate candidate change points to estimate change points and determine pulse segments from the change points.

Process 200 begins at act 210, where data is obtained, such as sequencing data 104 obtained by sequencer 102. Next, process 200 proceeds to act 220, where candidate change points within the data are evaluated, such as by using change point detector 110. If a change point has been detected by act 220, then process 200 proceeds to act 230, where candidate change points occurring in time prior to the detected change point is pruned, such as by using pruning methods 114. If no change point has been detected by act 220, then process 200 proceeds to act 210 where additional data is obtained and additional candidate change points are evaluated by act 220. In this manner, candidate change points are not removed from consideration as being change points if there is no change point detected and additional data is obtained until a change point is detected. In some embodiments, segment analyzer 116 may accumulate change points as they are being detected by change point detector 110. Additional discussion of statistical models and their parameters that may be used in evaluating candidate change points is described herein including, for example, with respect to FIG. 3A.

After pruning the prior candidate change points at act 240, process 200 proceeds to act 250 where the detected change points are accumulated as a set of change points for the data being obtained, such as change points 112. Next, process 200 proceeds to act 250, where individual pulse segments are determined using the accumulated change points, such as by using segment analyzer 116 to generate pulse segments 118. In some embodiments, some pulse segments for the data for a sequencing reaction may be determined prior to additional data for the sequencing reaction is obtained.

Next process 200 proceeds to act 260, where the pulse segments are passed to base caller, such as base caller 120, which may then use the pulse segments to determine output sequences for the data.

FIG. 3A is an exemplary trace of sequencing data, such as data obtained using sequencer 102. In the illustrated example, the sequencing data x_{s:e} starts from position s and ends at position e, and has a candidate change point m. Statistical models and their parameters may be used to determine whether the candidate change point m is an actual change point.

If the candidate change point m is not an actual change point, the log likelihood of the sequencing data may be generated by one distribution Θ_{s:e}. The probability of observing the sequencing data under this assumption is Pr(x_{s:e}|Θ_{s:e}). In an unsupervised regime that imposes no prior assumption to the parameter Θ_{s:e}, an estimate such as the maximum likelihood estimate (MLE) can be used to estimate the parameter Θ_{s:e}. That is

θ_{s:e}=MLE(x_{s:e}) (1)

The log likelihood of the sequencing data x_{s:e} given the parameter Θ_{s:e} is

$$ℒ ( {x}_{s : e \vert {θ}_{s : e}} ) = log Pr ( {x}_{s : e} \vert {θ}_{s : e} ) = {\sum }_{i = s}^{ɛ} log Pr ( {x}_{s} \vert {θ}_{s : e} ) \qquad ( 2 )$$

If the candidate change point m is an actual change point, the log likelihood of the sequencing data z_{s:e} may be generated by two different distributions characterized by the parameters Θ_{s:e} and Θ_{m+1:e}. The log likelihood of the sequencing data x_{s:e} under this assumption is

$$ℒ ( {x}_{s : e \vert {θ}_{s : m} , {θ}_{m + 1 , n}} ) = {\sum }_{i = s}^{m} log Pr ( {x}_{i} \vert {θ}_{s : m} ) + {\sum }_{i = m + 1}^{e} log Pr ( {x}_{i} \vert {θ}_{m + 1 : e} ) \qquad ( 3 )$$

By computing the difference between Equation (3) and Equation (2), the log likelihood that the candidate change point m is an actual change point is obtained as follow:

$$\begin{matrix}ℒ ( e = m ) = ( ℒ ( {x}_{s : e \vert {θ}_{s : m} , {θ}_{m + 1 , n}} ) - ℒ ( {x}_{s : e \vert {θ}_{s : e}} ) ) \\ = ( {\sum }_{i = s}^{m} log Pr ( {x}_{i} \vert {θ}_{s : m} ) + {\sum }_{i = m + 1}^{e} log Pr ( {x}_{i} \vert {θ}_{m + 1 : e} ) - {\sum }_{i = s}^{e} log Pr ( {x}_{i} \vert {θ}_{s : e} )\end{matrix} \qquad ( 4 )$$

In some embodiments, the candidate change point m may be determined to be an actual change point when its log likelihood given by Equation (4) is greater than a threshold value T.

The log likelihood that the candidate change point m is an actual change point may depend on the underlying probability distribution. In some embodiments, the underlying distribution may be univariate (e.g., for one time bin series data) and the sequencing data may follow a Poisson distribution, the MLE for the parameter Θ in equation (1) is: Θ_{s:e}=x̅_{s:e}, wherein x̅_{s:e} denotes the mean of data x_{s:e}. Assuming the data x_{s:e} belong to a same distribution, the log likelihood of the data x_{s:e} is

$$\begin{matrix}ℒ ( {x}_{s : e} \vert {θ}_{s : e} ) = {\sum }_{i = s}^{t} log Pr ( {x}_{i} \vert {θ}_{s : e} ) \\ = {\sum }_{i = s}^{e} ( - {θ}_{s : e} + {x}_{i} log ( {θ}_{s : e} ) - log ( {x}_{i} ! ) ) \\ = - ( e - s + 1 ) {\overline{x}}_{s : e} + ( t - e + 1 ) {x}_{s : e} log ( {\overline{x}}_{s : e} ) - {\sum }_{i = s}^{t} log ( {x}_{i} ! ) \\ = - {\sum }_{i = s}^{e} {x}_{i} + {\sum }_{i = s}^{t} {x}_{i} log ( {\overline{x}}_{s : e} ) - {\sum }_{i = s}^{e} log ( {x}_{i} ! )\end{matrix} \qquad ( 5 )$$

By applying Equation (5) to Equation (4), the log likelihood that the candidate change point m is an actual change point is obtained as follow:

$$\begin{matrix}ℒ ( c = m ) = - {\sum }_{i = s}^{m} {x}_{i} + {\sum }_{i = s}^{m} {x}_{i} log ( {\overline{x}}_{s : m} ) - {\sum }_{i = s}^{m} log ( {x}_{i} ! ) - {\sum }_{i = m + 1}^{e} {x}_{i} + \\ {\sum }_{i = m + 1}^{e} {x}_{i} log ( {\overline{x}}_{m + 1 , e} ) - {\sum }_{i = m + 1}^{m} log ( {x}_{i} ! ) + {\sum }_{i = s}^{e} {x}_{i} - \\ {\sum }_{i = s}^{e} {x}_{i} log ( {\overline{x}}_{s : e} ) + {\sum }_{i = s}^{e} log ( {x}_{i} ! ) \\ = {\sum }_{i = s}^{m} {x}_{i} log ( {\overline{x}}_{s : m} ) + {\sum }_{i = m + 1}^{e} {x}_{i} log ( {\overline{x}}_{m + 1 , e} ) - {\sum }_{i = s}^{e} {x}_{i} log ( {\overline{x}}_{s , n} )\end{matrix} \qquad ( 6 )$$

In some embodiments, the log likelihood of the sequencing data under a given hypothesis (e.g., a hypothetical distribution) is equivalent to the cost of encoding the data under that hypothesis. The cost of the sequencing data x_{s:e} can be efficiently computed as follows:

$$cost ( s , e ) = ( {𝒞}_{e} - {𝒞}_{s} ) log \frac{{𝒞}_{e} - {𝒞}_{s}}{e - s + 1} \qquad ( 7 )$$

wherein C₁ represents the accumulated sum of the data point and therefore [symbol] ᵢ=Σᵢ₌₁ⁱ×ᵢ.

In some embodiments, the underlying distribution may be a Gaussian distribution with known variance σ², then the problem is to find where the generating processes change the means of the sequencing data x_{s:e}. The MLE for the mean μ_{s:e} of such processes are itis

μ_{s:e}=x̅_{s:e} (8)

The log likelihood of the sequencing data x_{s:e} under this distribution is

$$\begin{matrix}ℒ ( {x}_{s : e} \vert {μ}_{s : e} , {σ}^{2} ) = {\sum }_{i = s}^{e} ( log \frac{1}{\sqrt{2 π {σ}^{2}}} - \frac{{( {x}_{i} - {μ}_{s : e} )}^{2}}{2 {σ}^{2}} ) \\ = ( e - s + 1 ) log \frac{1}{\sqrt{2 π {σ}^{2}}} - \frac{{\sum }_{i = s}^{e} {( {x}_{i} - {μ}_{s : e} )}^{2}}{2 {σ}^{2}} \\ = ( e - s + 1 ) log \frac{1}{\sqrt{2 π {σ}^{2}}} - \\ \frac{{\sum }_{i = s}^{e} {x}_{i}^{2}}{2 {σ}^{2}} + \frac{( e - s + 1 ) {μ}_{s : e}^{2}}{2 {σ}^{2}}\end{matrix} \qquad ( 9 )$$

By applying Equation (9) to Equation (4), the log likelihood that the candidate change point m is an actual change point is obtained as follow:

$$ℒ ( e = m ) = \frac{( m + s + 1 ) {μ}_{s : m}^{2}}{2 {σ}^{2}} + \frac{( e - m ) {μ}_{m + 1 : e}^{2}}{2 {σ}^{2}} - \frac{( e - s + 1 ) {μ}_{s : e}^{2}}{2 {σ}^{2}} \qquad ( 10 )$$

The cost of the sequencing data x_{s:e} can be efficiently computed as follow:

$$cost ( s , e ) = \frac{{( {𝒞}_{e} - {𝒞}_{s} )}^{2}}{2 ( e - s + 1 ) {σ}^{2}} \qquad ( 11 )$$

In some embodiments, a data point may be k-dimensional, where xᵢ^{j} follows a univariate distribution j for j∈1 . . . K. Example of this kind of data includes the two time bin series. Assuming that these components switch together but are independent of each other given their parameter during a homogeneous segment, the cost of a data segment may be computed as the sum of the costs of all components.

The inventors have recognized and appreciated that conventional change point detection techniques examine every possible candidate change point, and return the position that maximizes the log likelihood of Equation (4). These conventional change point detection techniques are computationally expensive because for every interval of a length len=e−s+1, these techniques have to evaluate Equation (4) for 4*len times. To identify multiple change points, these conventional change point detection techniques take the order of O(n²) computations and therefore are not tractable for long time series data.

The inventors have developed new change point detection techniques that allow change points to be detected with much less computational resources and therefore to be detected in real-time. In some embodiments, the log likelihood of the sequencing data under a given hypothesis (e.g., a hypothetical distribution) is equivalent to the cost of encoding the data under that hypothesis. In some embodiments, an adaptive encoding approach may be adopted by segmenting a time series data into data segments x_{c}_{₁}_{:c}_{₂},x_{c}_{₂}_{:c}_{₂}, . . . , x_{c}_{ₘ}_{-1:c}_{ₘ} where 1=c₁<c₂< . . . <cₘ=n and each data segment may be encoded with the cost specified by Equation (2). The best set of change points may be found by optimizing the segmentation such that the total cost of encoding the data is minimized.

An exemplary algorithm with such change point detection techniques is as follows:

Algorithm 1 Online changepoint algorithm

```
online_changepoint(x [symbol] _{..n}, [symbol] , T)
param l: Bound for looking back
param T: Threshold for specifying change point [symbol]
← empty array [symbol]
[0] ← 0
cost ← empty array
F ← empty array
last_change_point ← 0
for i ← 1 to |X| do
  receive(xᵢ) [symbol]
  [ [symbol] ] ← [symbol]  [i − 1] + x [symbol]
  cost[ [symbol] ] ← [symbol]  (xₗₐₛₜ_change_point:i)
  for all m ∈ {last_change_point} ∪{j : i − l ≤ j < i} do
    F cost[m] ← cost[m] + [symbol]  (m : [symbol] )
  end for
  Find \hat{m} ∈ {last_change_point} ∪{j : i − [symbol]  ≤ j < i} that maximizes F[\hat{m}]
  cost[i] ← F[\hat{m}] + T
  link[i] ← \hat{m}
end for [symbol]
indicates data missing or illegible when filed
```

As shown above, Algorithm 1 is configured to detect change points in the data x_{, . . . n}, which may be associated with at least one molecule undergoing a sequencing reaction. Algorithm 1 starts by obtaining data points xᵢ of the data x_{1 . . . n}. Algorithm 1 then computes the accumulated sum C[i] of the data points xᵢ and the cost function cost[i] of the data segment x_{last_change_point:i}. For each candidate change point m that is either the last identified change point or within the time window of position i−l to position i, Algorithm 1 computes a function F*cost[m] by combining the cost function cost[m] and the log likelihood [symbol] (m:i). As described below, to identify a change point, Algorithm 1 looks back at most l steps. In some embodiments, for each step, simple multiplication and division operations for the Gaussian distributions (see Equation (11)) may be computed. In some embodiments, for each step, a log operation for the Poisson distribution (see Equation (7)) may be computed. The computation of the log operation may be simplified by pre-computing approximate μ values and cache the pre-computed values in an array. Lastly, in the illustrated example, a change point \hat{m} is detected when the function F is maximized at \hat{m}. In some embodiments, the function F may be determined as being maximized when its value is above the threshold T.

It should be appreciated that Algorithm 1 provides the following characteristics. First, for each candidate change point m, it can be determined whether the candidate change point m is an actual change point after observing at most l data points following the candidate change point m. l is bounded by the signal-to-noise ratio (SNR) of the data. Therefore, Algorithm 1 takes the order of O(nl) computation in the worst case, and provides a smaller look back period and a faster algorithm with better SNR data.

The above-described first characteristic may be proved by assuming that the data before and after the candidate change point m follow the univariate Gaussian distributions with known variance σ² and with means Θ_{s:m} and Θ_{m+1:m+1}, respectively and that the candidate change point m is identified as a change point at a time point m+1 when the log likelihood is greater than the threshold T. Under these assumptions, Equation (10) may be used to compute the log likelihood as

$$ℒ ( e = m ) = \frac{( m + s + 1 ) {μ}_{s : m}^{2}}{2 {σ}^{2}} + \frac{( m + 1 - m ) {μ}_{m + 1 : m + l}^{2}}{2 {σ}^{2}} - \frac{( m + l - s + 1 ) {μ}_{κ : m + l}^{2}}{2 {σ}^{2}} \geq T \qquad ( 12 )$$

where μₛᵢₘ₊₁ is estimated as

$${μ}_{s : m + l} = \frac{( m - s + 1 ) {μ}_{s : m} + {l}_{{μ}_{m + 1 : m + l}}}{l - s + 1} \qquad ( 13 )$$

By substituting Equation (13) into Equation (12), the equation for l may be solved to obtain

$$\begin{matrix}\frac{1}{( m - s + 1 )} + \frac{1}{l} \leq \frac{{( {μ}_{s : m} - {μ}_{m + 1 : m + l} )}^{2}}{2 T {σ}^{2}} \\ or & ( 14 ) \\ t \geq \frac{1}{\frac{{( {μ}_{s : m} - {μ}_{m + 1 : m + l} )}^{2}}{2 T {σ}^{2}} - \frac{1}{( m - s + 1 )}} > 2 T \frac{{σ}^{2}}{{( {μ}_{s : m} - {μ}_{m + 1 : m + l} )}^{2}} & ( 15 )\end{matrix}$$

These results entail that at any time t, the condition specified in Equation (4) is needed to be computed for at most l times where l is

$$2 T \frac{{σ}^{2}}{{( {μ}_{s : m} - {μ}_{m + 1 : m + l} )}^{2}}$$

and therefore can be estimated from the characteristics of the data. This bound/is inversely proportional to the SNR where the SNR is defined by

$$SNR = \frac{{μ}_{m -} - {μ}_{m +}}{σ} ,$$

μₘ₋ and μₘ₊ are the means of the data before and after the change point m.

Second, when a change point at position m is identified, all the non-change points before n can be excluded from the possibility of being an actual change point and therefore can be pruned from the change point candidates. Therefore, with Algorithm 1, the finding of one change point in the interval can be extended to multiple change points.

Using the techniques described herein, a change point, m, has been identified. The data within the time prior to the change point, m, has a lower intensity than the data within the time occurring after the change point, m. In this instance, the time prior to the change point is indicative of background signal while the time after the change point is indicative of a pulse segment associated with a nucleotide incorporation event.

FIG. 3B is a schematic of detecting change points in sequencing data and generating an output sequence. As shown, sequencing data 300 is obtained, such as by using sequencer 102, to generate intensity measurements over time. In the context of FIG. 3B, sequencing data 300 is being obtained in real-time during a sequencing reaction and the vertical dashed line on the right shows the current time of the sequencing reaction. Change points (shown as the short vertical arrows below sequencing data 300) are detected, such as by using change point detector 110, during the sequencing reaction. In particular, change point detector 110 may maintain time window 320 of sequencing data 300 in memory and use the data in time window 320 in detecting the change points. Time window 320 may adjust along the time axis as change points are detected and as additional sequencing data 300 is obtained. Additional discussion of statistical models and their parameters that may be used in determining candidate change points is described herein including, for example, in relating to FIG. 3A.

The change points may then be used to determine pulse segments (shown as the horizontal lines under the change points) of sequencing data 300 corresponding to pulses, such as by using segment analyzer 116. This process may be referred to as “pulse calling.” As shown in FIG. 3B, segment analyzer 116 may maintain recent change points, such as change points within time window 340, in memory and use those change points to call pulses. In some instances, time window 340 is larger than time window 320, such as when there are fewer change points to store in memory than the raw sequencing data 300.

Change point detector 110 may detect individual change points and pass those change points to segment analyzer 116. In some instances, some of the data associated with the detected change points may also be passed to segment analyzer 116 and used to perform pulse calling. In some embodiments, detected change points and features of the data between successive change points may be passed to segment analyzer 116. For example, a distance between two successive change points may be passed to segment analyzer 116 and used to determine pulse segments. As another example, a mean value and a standard deviation value of the data between successive change points may be passed to segment analyzer 116 and used to determine pulse segments. In this manner, change point detector 110 may be configured to compress the raw sequencing data, thus reducing the amount of raw sequencing data needed to be maintained in memory to perform real-time analysis. In addition, time window 340 may correspond to a longer duration of time than time window 320 since not all of the raw sequencing data corresponding to time window 340 is maintained in memory.

The pulse segments may then be used to determine bases, such as by using base caller 120. In particular, base caller 120 may analyze the pulse segments within time window 360 to determine output bases, such as bases “A,” “C,” “T,” and “G” shown in FIG. 3B. Time window 360 may be longer than time window 340, in some embodiments. Similar to segment analyzer 116, base caller 120 may receive features of the data corresponding to individual pulse segments from segment analyzer 116. For example, base caller 120 may receive distances corresponding to individual pulse segments from segment analyzer 116 and use the distances to perform base calling. As another example, base caller 120 may receive a mean value and a standard deviation value corresponding to individual pulse segments from segment analyzer 116 and use those values to perform base calling. Using such features associated with pulse segments within time window 360 may allow for base caller 120 to reduce the amount of memory needed to perform base calling. In particular, not all of the raw sequencing data corresponding to time window 360 may be stored in the memory, which may allow for time window 360 to correspond to a longer duration of time than time window 320 and/or time window 340.

Limiting the amount of data maintained in memory to the data corresponding to windows 320, 340, and 360 may reduce the memory footprint needed to perform sequencing analysis and sequencing data 300. This reduction of memory needed may allow for change point detection, pulse calling, and base calling to be performed while additional sequencing data 300 is obtained. In addition, the output bases can be outputted during the sequencing reaction on a short time-scale (e.g., on the order of seconds).

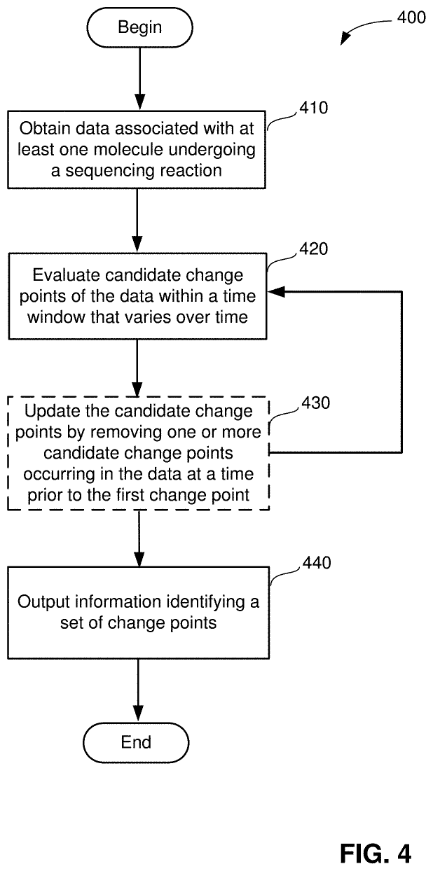

FIG. 4 is a flow chart of an illustrative process 400 for evaluating candidate change points, in accordance with some embodiments of the technology described herein. Process 400 may be performed on any suitable computing device(s) (e.g., a single computing device, multiple computing devices co-located in a single physical location or located in multiple physical locations remote from one another, one or more computing devices part of a cloud computing system, etc.), as aspects of the technology described herein are not limited in this respect. In some embodiments, sequencer 102, preprocessing methods 106, change point detector 110, pruning methods 114, and segment analyzer 116 may perform some or all of process 400 to obtain sequencing data and evaluate candidate change points.

Process 400 begins at act 410, where data regarding light detected over time from luminescent labels associated with at least one molecule undergoing a sequencing reaction is obtained, such as by using sequencer 102. In some embodiments, obtaining the data includes receiving portions of the data at different times. In some embodiments, obtaining the data occurs during the sequencing reaction. The data may be associated with one or more molecules undergoing the sequencing reaction. In some embodiments, the luminescent labels correspond to nucleotides being incorporated into a nucleotide sequence during the sequencing reaction. In some embodiments, the luminescent labels correspond to amino acid sequences of a peptide. In some embodiments, obtaining the data further comprises obtaining data identifying an intensity characteristic of the light and a temporal characteristic of the light. The temporal characteristic may include a ratio of photons detected in different time bins.

Next, process 400 proceeds to act 420, where candidate change points of the data within a time window of the data that varies over time are evaluated, such as by using change point detector 110 and pruning methods 114. In embodiments where portions of the data are received at different times, the time window of the data may be adjusted after receiving one or more portions of the data. Some embodiments may involve performing evaluation of candidate change points at least in part by evaluating candidate change points of the data within a first time window of the data, generating a second time window at least in part by adding, to the first time window, one or more data points occurring after the first time window, and evaluating candidate change points of the data within the second time window.

In some embodiments, evaluating the candidate change points may involve estimating a change point of the data and evaluating candidate change points of the data occurring after the change point. In such embodiments, evaluating the candidate change points may involve using one or more portions of data occurring prior to the change point. According to some embodiments, evaluating the candidate change points may include estimating a set of change points. The set of change points may be used in determining one or more nucleotide incorporation events, such as by using segment analyzer 116 and base caller 120 to determine one or more output sequences. In embodiments that involve obtaining data associated with one or more molecules undergoing a sequencing reaction, evaluating the candidate change points may include estimating a set of change points of data associated with individual molecules. For example, a first set of change points may be estimated for a first molecule undergoing a sequencing reaction, and a second set of change points may be estimated for a second molecule undergoing a sequencing reaction.

Some embodiments involve evaluating the candidate change points by estimating a first change point of the data by evaluating a first set of candidate change points and estimating a second change point of the data by evaluating a second set of candidate change points occurring after the first change point. In some embodiments, estimating the first change point further includes evaluating the first set of candidate change points using first data and estimating the second change point further includes evaluating the second set of candidate change points using second data that includes at least a portion of the first data. In some embodiments, the second set of candidate change points includes one or more candidate change points of the first set occurring after the first change point.

In some embodiments, evaluating the candidate change points is performed at least in part by using one or more statistical models that estimate the likelihood of the candidate change points being change points. In such embodiments, the one or more statistical models may be applied to the time window to estimate one or more change points.

In some embodiments, process 400 may involve updating the candidate change points based on one or more estimated change points and re-evaluating the updated candidate change points. In such embodiments, process 400 may proceed to act 430, where, after estimating a first change point of the set of change points, the candidate change points are updated by removing one or more candidate change points occurring in the data at a time prior to the first change point, such as by using pruning methods 114. The updated candidate change points may be re-evaluated by performing act 420 using the updated candidate change points. In some embodiments, re-evaluating the updated candidate change points further comprise estimating a second change point. The candidate change points may be updated by removing one or more candidate change points occurring in the data at a time prior to the second change point.

Next, process 400 proceeds to act 440, where an indication of information identifying a set of change points is output, such as to a user via a user interface.

Process 400 may include one more additional acts that involve using the set of change points. In some embodiments, process 400 may include determining segments of the data between individual change points of the set of change points as being a background signal. In some embodiments, process 400 may include determining segments of the data between individual change points of the set of change points as being nucleotide incorporation events.

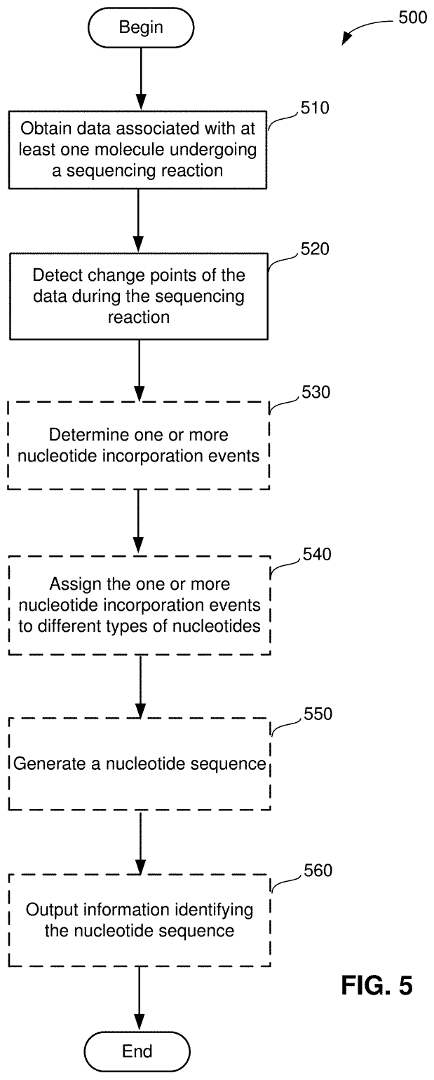

FIG. 5 is a flow chart of an illustrative process 500 for detecting change points during a sequencing reaction, in accordance with some embodiments of the technology described herein. Process 500 may be performed on any suitable computing device(s) (e.g., a single computing device, multiple computing devices co-located in a single physical location or located in multiple physical locations remote from one another, one or more computing devices part of a cloud computing system, etc.), as aspects of the technology described herein are not limited in this respect. In some embodiments, sequencer 102, preprocessing methods 106, change point detector 110, pruning methods 114, and segment analyzer 116 may perform some or all of process 500 to obtain data associated with one or more molecules undergoing a sequencing reaction and detect change points of the data during the sequencing reaction.

Process 500 begins at act 510, where data regarding light detected over time from luminescent labels associated with one or more molecules undergoing a sequencing reaction is obtained, such as by using sequencer 102. In some embodiments, the sequencing reaction is a primer extension reaction. In some embodiments, the data is indicative of light detected from luminescent labels during one or more nucleotide incorporation events. The luminescent labels may correspond to nucleotides being incorporated into a nucleotide sequence during the sequencing reaction. In some embodiments, obtaining the data may include receiving portions of the data at different times.

In some embodiments, obtaining the data may include obtaining data identifying an intensity characteristic of the light and a temporal characteristic of the light. The temporal characteristic may include a ratio of photons detected in different time bins.

In some embodiments, obtaining the data may include obtaining time-bin information regarding the times at which the luminescent labels emit light in response to excitations of the luminescent labels. In such embodiments, obtaining the data may include calculating light intensity information based on the time-bin information and using the light intensity information in detecting change points. In some embodiments, calculating the light intensity information may include summing the time-bin information for individual times of the data.

Next, process 500 proceeds to act 520, where change points of the data during sequencing are detected, such as by using change point detector 110 and pruning methods 114. Detecting the change points may involve estimating a set of candidate change points and evaluating the set of candidate change points. In some embodiments, act 520 may be performed by a sequencing instrument, such as sequencer 102.

In some embodiments, detecting the change points may include detecting a first change point of the data and determining candidate change points of the data occurring after the first change point. In such embodiments, determining candidate change points of the data occurring after the first change point may include using one or more portions of data occurring prior to the first change point. The candidate change points occurring after the first change point may then be used to detect a second change point, such as by selecting one of the candidate change points as being the second change point. This process may be repeated to detect a set of change points. For example, the process may involve determining candidate change points of the data occurring after the second change point and detecting a third change point based on the candidate change points.

In some embodiments, detecting change points is performed at least in part by using one or more statistical models having a parameter representing the likelihood of having a change point at a particular time. In such embodiments, detecting the change points further involves using the data as an input to the one or more statistical models to determine a set of candidate change points as an output of the one or more statistical models. Detecting the change points may be based on the set of candidate change points.

In some embodiments, obtaining the data at act 510 may include obtaining data associated with multiple molecules undergoing a sequencing reaction, and detecting change points at act 520 may include detecting a set of change points of data associated with the individual multiple molecules.

In some embodiments, process 500 proceeds to act 530, where one or more nucleotide incorporation events are determined at least in part based on detecting the change points. In particular, regions between successive change points may be determined as being associated with nucleotide incorporation events or background signal.

In some embodiments, act 530 may involve determining segments of the data between individual change points as corresponding to a background signal. In such embodiments, determining the segments of the data may include comparing values of one or more features of the data within the segments to a threshold value, and identifying segments having values of the one or more features below the threshold value as corresponding to the background signal.

In some embodiments, act 530 may involve determining segments of the data between individual change points of a set of change points as being nucleotide incorporation events. In such embodiments, determining the segments of the data may include comparing values of one or more features of the data within the segments to a threshold value, and identifying segments having values of the one or more features above the threshold value as corresponding to nucleotide incorporation events.

In some embodiments, process 500 proceeds to act 540, where the one or more nucleotide incorporation events are assigned to different types of nucleotides. Process 500 may proceed to act 550, where a nucleotide sequence is generated based on the assigning performed in act 540.

Next, process 500 proceeds to act 560, where an indication of information identifying the nucleotide sequence is output, such as to a user via a user interface.

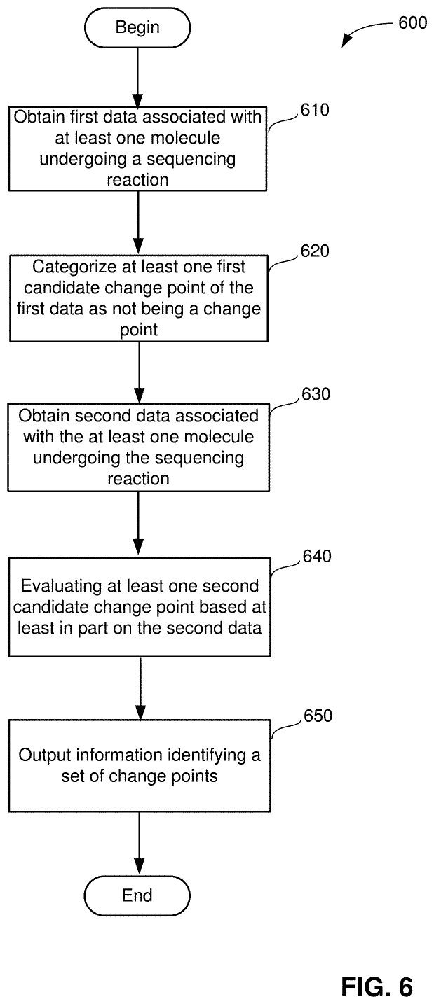

FIG. 6 is a flow chart of an illustrative process 600 for evaluating candidate change points, in accordance with some embodiments of the technology described herein. Process 600 may be performed on any suitable computing device(s) (e.g., a single computing device, multiple computing devices co-located in a single physical location or located in multiple physical locations remote from one another, one or more computing devices part of a cloud computing system, etc.), as aspects of the technology described herein are not limited in this respect. In some embodiments, sequencer 102, preprocessing methods 106, change point detector 110, pruning methods 114, and segment analyzer 116 may perform some or all of process 400 to obtain data, categorize candidate change point(s), and evaluate the candidate change point(s).

Process 600 begins at act 610, where first data associated with one or more molecules undergoing a sequencing reaction is obtained.

Next, process 600 proceeds to act 620, where one or more first candidate change points of the first data are categorized as not being a change point. In some embodiments, categorizing the one or more first candidate change points includes determining that a change point does not exist within the first data. In some embodiments, obtaining the first data includes obtaining data identifying an intensity characteristic of the light and a temporal characteristic of the light. The temporal characteristic may include a ration of photons detected in different time bins.

In some embodiments, categorizing the one or more first candidate change points is performed at least in part by using one or more statistical models having a parameter representing a likelihood of having a change point at a particular time. In such embodiments, categorizing the one or more first candidate change points may include using the one or more statistical models to calculate one or more scores for the one or more first candidate change points and using the one or more scores to determine that the one or more first candidate change point is not a change point. In some embodiments, categorizing the one or more first candidate change points may include calculating one or more values of the parameter associated with the one or more first candidate change points and using the one or more values to determine that the one or more first candidate change points are not change points. In such embodiments, categorizing the one or more first candidate change points further includes comparing the one or more values to a threshold value. In some embodiments, evaluating the one or more second candidate change points includes using one or more statistical to calculate one or more scores for the one or more second candidate change points and using the one or more scores to evaluate the one or more second candidate change points. In some embodiments, evaluating the one or more second candidate change points includes calculating one or more values of the parameter associated with the one or more second candidate change points and using the one or more values to evaluate the one or more second candidate change points.

Next, process 600 proceeds to act 630, where second data associated with the one or more molecules undergoing the sequencing reaction is obtained. In some embodiments, the second data occurs in time after the first data.

Next, process 600 proceeds to act 640, where one or more second candidate change points is evaluated based at least in part on the second data. In some embodiments, evaluating the one or more second candidate change points is performed based on one or more portions of the first data and the second data. In some embodiments, evaluating the one or more second candidate change points includes categorizing one or more data points of the one or more second candidate change points as being a change point. In such embodiments, process 600 may include determining one or more nucleotide incorporation events based at least in part on the one or more data points.

Next process 600 proceeds to act 650, where an indication of information identifying a set of change points is output, such as to a user via a user interface.

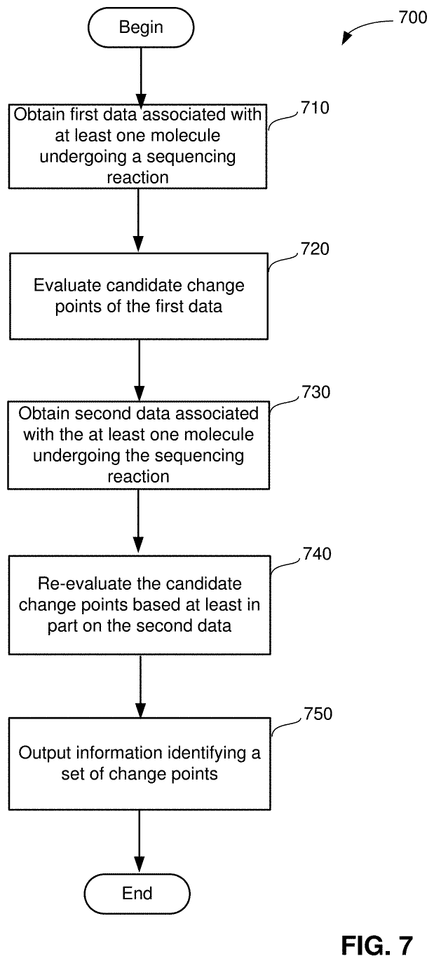

FIG. 7 is a flow chart of an illustrative process 700 for evaluating candidate change points, in accordance with some embodiments of the technology described herein. Process 700 may be performed on any suitable computing device(s) (e.g., a single computing device, multiple computing devices co-located in a single physical location or located in multiple physical locations remote from one another, one or more computing devices part of a cloud computing system, etc.), as aspects of the technology described herein are not limited in this respect. In some embodiments, sequencer 102, preprocessing methods 106, change point detector 110, pruning methods 114, and segment analyzer 116 may perform some or all of process 600 to obtain data and evaluate candidate change points of the data.

Process 700 begins at act 710, where first data regarding light detected over time from luminescent labels associated with one or more molecules undergoing a sequencing reaction is obtained. In some embodiments, obtaining the data may include obtaining data identifying an intensity characteristic of the light and a temporal characteristic of the light. The temporal characteristic may be a ratio of photons detected in different time bins.

Next, process 700 proceeds to act 720, where candidate change points of the first data are evaluated. Evaluating the candidate change points of the first data may involve determining that a change point does not exist within the first data.

In some embodiments, evaluating the candidate change points of the first data is performed at least in part by using one or more statistical models having a parameter representing a likelihood of having a change point at a particular time. In such embodiments, evaluating the candidate change points of the first data may include using the one or more statistical models to calculate scores for the candidate change points based on the first data. Evaluating the candidate change points may include comparing the scores to a threshold value. In some embodiments, evaluating the candidate change points may include calculating values of the parameter associated with the candidate change points based on the first data.

Next, process 700 proceeds to act 730, where second data regarding light detected from one or more luminescent labels associated with the one or more molecules undergoing the sequencing reaction is obtained. The second data may occur in time after the first data.

Next, process 700 proceeds to act 740, the candidate change points are re-evaluated based at least in part on the second data. Re-evaluating the candidate change points may be based at least in part on one or more portions of the first data and the second data. In some embodiments, re-evaluating the candidate change points may involve estimating one or more change points. In such embodiments, process 700 may include determining one or more nucleotide incorporation events based on the one or more change points.

In some embodiments, re-evaluating the candidate change points is performed at least in part by using one or more statistical models having a parameter representing a likelihood of having a change point at a particular time. In such embodiments, re-evaluating the candidate change points may include using the one or more statistical models to calculate scores for the candidate change points based on one or more portions of the first data and the second data. Evaluating the candidate change points may include comparing the scores to a threshold value. In some embodiments, re-evaluating the candidate change points may include calculating values of the parameter associated with the candidate change points based on one or more portions of the first data and the second data.

Next, process 700 proceeds to act 750, where an indication of information identifying a set of change points is output, such as to a user via a user interface.

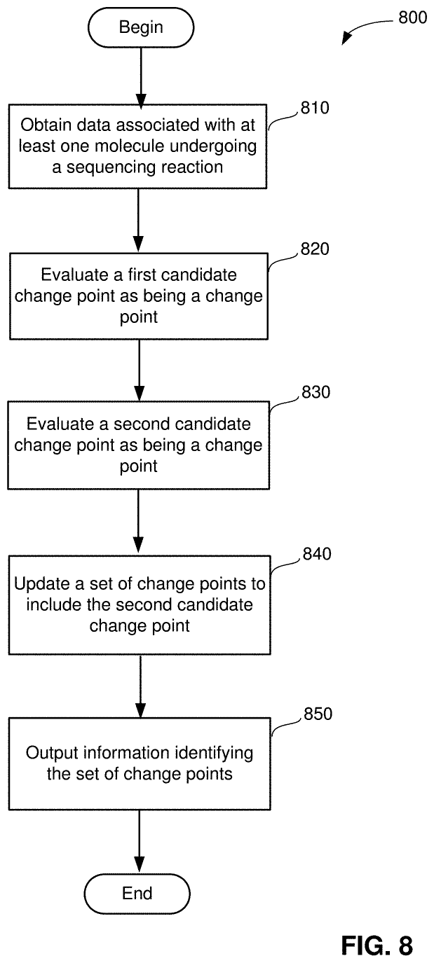

FIG. 8 is a flow chart of an illustrative process 800 for evaluating candidate change points, in accordance with some embodiments of the technology described herein. Process 800 may be performed on any suitable computing device(s) (e.g., a single computing device, multiple computing devices co-located in a single physical location or located in multiple physical locations remote from one another, one or more computing devices part of a cloud computing system, etc.), as aspects of the technology described herein are not limited in this respect. In some embodiments, sequencer 102, preprocessing methods 106, change point detector 110, pruning methods 114, and segment analyzer 116 may perform some or all of process 400 to predict chemical reaction(s) and output molecule(s).

Process 800 begins at act 810, where data regarding light detected over time from luminescent labels associated with one or more molecules undergoing a sequencing reaction is obtained. In some embodiments, obtaining the data occurs during the sequencing reaction.

Next, process 800 proceeds to act 820, where a first candidate change point of the data is evaluated as being a change point. In some embodiments, evaluating the first candidate change point is performed at least in part by using one or more statistical models having a parameter representing a likelihood of having a change point at a particular time. In such embodiments, evaluating the first candidate change point may include using the one or more statistical models to calculate a score for the first candidate change point. Evaluating the first candidate change point may include calculating a value of the parameter for the first candidate change point and calculating the score based on the value of the parameter. In some embodiments, evaluating the first candidate change point may include calculating values of the parameter associated with different data points within the data and determining the first candidate change point is a change point based on the values.

Next, process 800 proceeds to act 830, where, for a portion of the data occurring at a time after the first candidate change point, a second candidate change point is evaluated as being a change point. In some embodiments, evaluating the second candidate change point may include evaluating candidate change points occurring at times after the first candidate change point.

In some embodiments, evaluating the second candidate change points is performed at least in part by using one or more statistical models. In such embodiments, evaluating the second candidate change point may include using the one or more statistical models to calculate a score for the second candidate change point. Evaluating the second candidate change point may include calculating a value of the parameter for the second candidate change point and calculating the score based on the value of the parameter. In some embodiments, evaluating the second candidate change point may include calculating values of the parameter associated with different times occurring after the first candidate change point and determining the second candidate change point is a change point based on the values.

Next, process 800 proceeds to act 840, where a set of change points is updated to include the second candidate change point. In some embodiments, process 800 may include determining one or more nucleotide incorporation events based on the set of change points. In some embodiments, process 800 may include determining segments of the data between individual change points of the set of change points as being nucleotide incorporation events. In some embodiments, process 800 may include determining segments of the data between individual change points of the set of change points as being a background signal.

Next, process 800 proceeds to act 850, where an indication of information identifying the set of change points is output, such as to a user via a user interface.

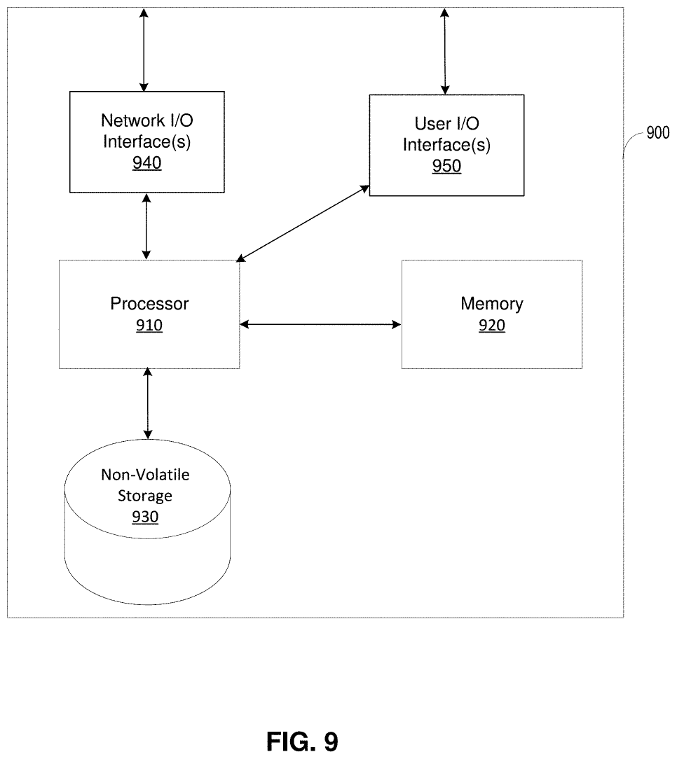

An illustrative implementation of a computer system 900 that may be used in connection with any of the embodiments of the technology described herein is shown in FIG. 9. The computer system 900 includes one or more processors 910 and one or more articles of manufacture that comprise non-transitory computer-readable storage media (e.g., memory 920 and one or more non-volatile storage media 930). The processor 910 may control writing data to and reading data from the memory 920 and the non-volatile storage device 930 in any suitable manner, as the aspects of the technology described herein are not limited in this respect. To perform any of the functionality described herein, the processor 910 may execute one or more processor-executable instructions stored in one or more non-transitory computer-readable storage media (e.g., the memory 920), which may serve as non-transitory computer-readable storage media storing processor-executable instructions for execution by the processor 910.

Computing device 900 may also include a network input/output (I/O) interface 940 via which the computing device may communicate with other computing devices (e.g., over a network), and may also include one or more user I/O interfaces 950, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

Examples that may be implemented according to some embodiments include the following:

A1. A method, comprising:

obtaining data regarding light detected over time from luminescent labels associated with at least one molecule undergoing a sequencing reaction;

evaluating candidate change points of the data within a time window of the data that varies over time; and outputting information identifying a set of change points based on evaluating the candidate change points.

A2. The method of Example A1, wherein obtaining the data further comprises receiving portions of the data at different times.
A3. The method of Example A2, wherein the method further comprises, after receiving at least one portion of the data, adjusting the time window of the data to include the at least one portion of data.
A4. The method of Example A1, wherein evaluating the candidate change points further comprises estimating a change point of the data and evaluating candidate change points of the data occurring after the change point.
A5. The method of Example A4, wherein evaluating candidate change points of the data occurring after the change point further comprises using at least a portion of data occurring prior to the change point.
A6. The method of Example A1, wherein evaluating the candidate change points is performed at least in part by:

evaluating candidate change points of the data within a first time window of the data;

generating a second time window at least in part by adding, to the first time window, at least one data point occurring after the first time window; and

evaluating candidate change points of the data within the second time window.

A7. The method of Example A1, wherein evaluating the candidate change points is performed at least in part by:

estimating a first change point of the data by evaluating a first set of candidate change points; and

estimating a second change point of the data by evaluating a second set of candidate change points occurring after the first change point.

A8. The method of Example A7, wherein estimating the first change point further comprises evaluating the first set of candidate change points using first data and estimating the second change point further comprises evaluating the second set of candidate change points using second data that includes at least a portion of the first data.
A9. The method of Example A7, wherein the second set of candidate change points includes at least one candidate change point of the first set occurring after the first change point.
A10. The method of Example A1, wherein evaluating the candidate change points is performed at least in part by using at least one statistical model that estimates the likelihood of the candidate change points being change points.
A11. The method of Example A10, wherein evaluating candidate change points further comprises applying the at least one statistical model to the time window to estimate at least one change point.
A12. The method of Example A1, wherein evaluating candidate change points further comprises estimating a set of change points.
A13. The method of Example A12, wherein the method further comprises determining at least one nucleotide incorporation event using the set of change points.
A14. The method of Example A12, wherein the method further comprises:

after estimating a first change point of the set of change points, updating the candidate change points by removing at least one candidate change point occurring in the data at a time prior to the first change point; and

re-evaluating the updated candidate change points.

A15. The method of Example A14, wherein re-evaluating the updated candidate change points further comprises estimating a second change point.
A16. The method of Example A14, wherein the method further comprises determining segments of the data between individual change points of the set of change points as being background signal.
A17. The method of Example A14, wherein the method further comprises determining segments of the data between individual change points of the set of change points as being nucleotide incorporation events.
A18. The method of Example A1, wherein obtaining the data occurs during the sequencing reaction.
A19. The method of Example A1, wherein the luminescent labels correspond to nucleotides being incorporated into a nucleotide sequence during the sequencing reaction.
A20. The method of Example A1, wherein the luminescent labels correspond to amino acids of a peptide.
A21. The method of Example A1, wherein obtaining the data further comprises obtaining data identifying an intensity characteristic of the light and a temporal characteristic of the light.
A22. The method of Example A21, wherein the temporal characteristic comprises a ratio of photons detected in different time bins.
A23. The method of Example A1, wherein obtaining the data further comprises obtaining data associated with a plurality of molecules undergoing a sequencing reaction, and evaluating the candidate change points further comprises estimating a set of change points of data associated with individual molecules of the plurality of molecules.
A24. A system comprising:

at least one hardware processor; and

at least one non-transitory computer-readable storage medium storing processor-executable instructions that, when executed by the at least one hardware processor, cause the at least one hardware processor to perform the method of any one of Examples A1-A23.

A25. At least one non-transitory computer-readable storage medium storing processor-executable instructions that, when executed by at least one hardware processor, cause the at least one hardware processor to perform the method of any one of Examples A1-A23.
A26. A sequencing instrument, comprising:

at least one photodetector configured to receive light from luminescent labels during a sequencing reaction;

at least one hardware processor; and

at least one non-transitory computer-readable storage medium storing processor-executable instructions that, when executed by the at least one hardware processor, cause the at least one hardware processor to perform the method of any one of Examples A1-A23.

B1. A method, comprising:

obtaining data regarding light detected over time from luminescent labels associated with at least one molecule undergoing a sequencing reaction; and

detecting change points of the data during the sequencing reaction.

B2. The method of Example B1, wherein the method further comprises determining at least one nucleotide incorporation event at least in part based on the detected change points.
B3. The method of Example B2, wherein the method further comprises:

assigning the at least one nucleotide incorporation event to different types of nucleotides;

generating, based on the assigning, a nucleotide sequence; and

outputting information identifying the nucleotide sequence.

B4. The method of Example B1, wherein detecting change points further comprises evaluating candidate change points of the data during the sequencing reaction.
B5. The method of Example B1, wherein the method further comprises determining at least one nucleotide incorporation event based on the detected change points.
B6. The method of Example B1, wherein the method further comprises determining segments of the data between individual change points as corresponding to background signal.
B7. The method of Example B6, wherein determining the segments of the data further comprises comparing values of at least one feature of the data within the segments to a threshold value, and identifying segments having values of the at least one feature below the threshold value as corresponding to background signal.
B8. The method of Example B1, wherein the method further comprises determining segments of the data between individual change points as being nucleotide incorporation events.
B9. The method of Example B8, wherein determining the segments of the data further comprises comparing values of at least one feature of the data within the segments to a threshold value, and identifying segments having values of the at least one feature above the threshold value as corresponding to nucleotide incorporation events.
B10. The method of Example B1, wherein the method further comprises outputting information identifying the change points.
B11. The method of Example B1, wherein the sequencing reaction is a primer extension reaction.
B12. The method of Example B1, wherein the data is indicative of light detected from luminescent labels during at least one nucleotide incorporation event.
B13. The method of Example B1, wherein obtaining the data further comprises receiving portions of the data at different times.
B14. The method of Example B1, wherein detecting change points further comprises detecting a first change point and determining candidate change points of the data occurring after the first change point.
B15. The method of Example B14, wherein determining candidate change points of the data occurring after the first change point further comprises using at least a portion of data occurring prior to the first change point.
B16. The method of Example B1, wherein detecting change points is performed at least in part by using at least one statistical model having a parameter representing the likelihood of having a change point at a particular time.
B17. The method of Example B16, wherein detecting change points further comprises using the data as an input to the at least one statistical model to determine a set of candidate change points as an output.
B18. The method of Example B1, wherein the luminescent labels correspond to nucleotides being incorporated into a nucleotide sequence during the sequencing reaction.
B19. The method of Example B1, wherein obtaining the data further comprises obtaining data identifying an intensity characteristic of the light and a temporal characteristic of the light.
B20. The method of Example B19, wherein the temporal characteristic comprises a ratio of photons detected in different time bins.
B21. The method of Example B1, wherein obtaining the data further comprises obtaining time-bin information regarding the times at which the luminescent labels emit light in response to excitations of the luminescent labels.
B22. The method of Example B21, wherein obtaining the data further comprises calculating light intensity information based on the time-bin information and using the light intensity information in evaluating the candidate change points.
B23. The method of Example B22, wherein calculating the light intensity information further comprises summing the time-bin information for individual times of the data.
B24. The method of Example B1, wherein obtaining the data further comprises obtaining data associated with a plurality of molecules undergoing a sequencing reaction, and detecting change points further comprises detecting a set of change points of data associated with the individual molecules of the plurality of molecules.
B25. The method of Example B1, wherein the detecting change points of the data is performed by a sequencing instrument.
B26. A system comprising:

at least one hardware processor; and

at least one non-transitory computer-readable storage medium storing processor-executable instructions that, when executed by the at least one hardware processor, cause the at least one hardware processor to perform the method of any one of Examples B1-B25.

B27. At least one non-transitory computer-readable storage medium storing processor-executable instructions that, when executed by at least one hardware processor, cause the at least one hardware processor to perform the method of any one of Examples B1-B25.
B28. A sequencing instrument, comprising:

at least one photodetector configured to receive light from luminescent labels during a sequencing reaction;

at least one hardware processor; and

at least one non-transitory computer-readable storage medium storing processor-executable instructions that, when executed by the at least one hardware processor, cause the at least one hardware processor to perform the method of any one of Examples B1-B25.

C1. A method, comprising:

obtaining first data regarding light detected from at least one luminescent label associated with at least one molecule undergoing a sequencing reaction;

categorizing at least one first candidate change point of the first data as not being a change point;

after the categorizing, obtaining second data regarding light detected from at least one luminescent label associated with the at least one molecule undergoing the sequencing reaction; and

evaluating at least one second candidate change point based at least in part on the second data.

C2. The method of Example C1, wherein categorizing the at least one first candidate change point further comprises determining that a change point does not exist within the first data.
C3. The method of Example C1, wherein categorizing the at least one first candidate change point is performed at least in part by using at least one statistical model having a parameter representing a likelihood of having a change point at a particular time.
C4. The method of Example C3, wherein categorizing the at least one first candidate change point further comprises using the at least one statistical model to calculate at least one score for the at least one first candidate change point and using the at least one score to determine that the at least one first candidate change point is not a change point.
C5. The method of Example C3, wherein categorizing the at least one first candidate change point further comprises calculating at least one value of the parameter associated with the at least one first candidate change point and using the at least one value to determine that the at least one first candidate change point is not a change point.
C6. The method of Example C5, wherein categorizing the at least one first candidate change point further comprises comparing the at least one value to a threshold value.
C7. The method of Example C3, wherein evaluating the at least one second candidate change point further comprises using the at least one statistical model to calculate at least one score for the at least one second candidate change point and using the at least one score to evaluate the at least one second candidate change point.
C8. The method of Example C3, wherein evaluating the at least one second candidate change point further comprises calculating at least one value of the parameter associated with the at least one second candidate change point and using the at least one value to evaluate the at least one second candidate change point.
C9. The method of Example C1, wherein the second data occurs in time after the first data.
C10. The method of Example C1, wherein evaluating at least one second candidate change point is performed based on at least a portion of the first data and the second data.
C11. The method of Example C1, wherein evaluating the at least one second candidate change point further comprises categorizing at least one data point of the at least one second candidate change point as being a change point.
C12. The method of Example C11, wherein the method further comprises determining at least one nucleotide incorporation event based at least in part on the at least one data point.
C13. The method of Example C1, wherein obtaining the first data further comprises obtaining data identifying an intensity characteristic of the light and a temporal characteristic of the light.
C14. The method of Example C13, wherein the temporal characteristic comprises a ratio of photons detected in different time bins.
C15. A system comprising:

at least one hardware processor; and

at least one non-transitory computer-readable storage medium storing processor-executable instructions that, when executed by the at least one hardware processor, cause the at least one hardware processor to perform the method of any one of Examples C1-C14.

C16. At least one non-transitory computer-readable storage medium storing processor-executable instructions that, when executed by at least one hardware processor, cause the at least one hardware processor to perform the method of any one of Examples C1-C14.
C17. A sequencing instrument, comprising.

at least one photodetector configured to receive light from luminescent labels during a sequencing reaction;

at least one hardware processor; and

at least one non-transitory computer-readable storage medium storing processor-executable instructions that, when executed by the at least one hardware processor, cause the at least one hardware processor to perform the method of any one of Examples C1-C14.

D1. A method, comprising:

obtaining first data regarding light detected over time from luminescent labels associated with at least one molecule undergoing a sequencing reaction;

evaluating candidate change points of the first data;

obtaining second data regarding light detected from at least one luminescent label associated with the at least one molecule undergoing the sequencing reaction; and re-evaluating the candidate change points based at least in part on the second data.

D2. The method of Example D1, wherein evaluating candidate change points of the first data further comprises determining that a change point does not exist within the first data.
D3. The method of Example D1, wherein re-evaluating the candidate change points is based at least in part on at least a portion of the first data and the second data.
D4. The method of Example D1, wherein the second data occurs in time after the first data.
D5. The method of Example D1, wherein re-evaluating the candidate change points further comprises estimating at least one change point.
D6. The method of Example D5, wherein the method further comprises determining at least one nucleotide incorporation event based on the at least one change point.
D7. The method of Example D1, wherein evaluating the candidate change points of the first data is performed at least in part by using at least one statistical model having a parameter representing a likelihood of having a change point at a particular time.
D8. The method of Example D7, wherein evaluating the candidate change points of the first data further comprises using the at least one statistical model to calculate scores for the candidate change points based on the first data.
D9. The method of Example D8, wherein evaluating the candidate change points further comprises comparing the scores to a threshold value.
D10. The method of Example D7, wherein evaluating the candidate change points further comprises calculating values of the parameter associated with the candidate change points based on the first data.
D11. The method of Example D1, wherein re-evaluating the candidate change points is performed at least in part by using at least one statistical model having a parameter representing a likelihood of having a change point at a particular time.
D12. The method of Example D11, wherein re-evaluating the candidate change points further comprises using the at least one statistical model to calculate scores for the candidate change points based on at least a portion of the first data and the second data.
D13. The method of Example D12, wherein evaluating the candidate change points further comprises comparing the scores to a threshold value.
D14. The method of Example D11, wherein re-evaluating the candidate change points further comprises calculating values of the parameter associated with the candidate change points based on at least a portion of the first data and the second data.
D15. The method of Example D1, wherein obtaining the data further comprises obtaining data identifying an intensity characteristic of the light and a temporal characteristic of the light.
D16. The method of Example D15, wherein the temporal characteristic comprises a ratio of photons detected in different time bins.
D17. A system comprising:

at least one hardware processor; and

at least one non-transitory computer-readable storage medium storing processor-executable instructions that, when executed by the at least one hardware processor, cause the at least one hardware processor to perform the method of any one of Examples D1-D16.

D18. At least one non-transitory computer-readable storage medium storing processor-executable instructions that, when executed by at least one hardware processor, cause the at least one hardware processor to perform the method of any one of Examples D1-D16.
D19. A sequencing instrument, comprising:

at least one photodetector configured to receive light from luminescent labels during a sequencing reaction;

at least one hardware processor; and

at least one non-transitory computer-readable storage medium storing processor-executable instructions that, when executed by the at least one hardware processor, cause the at least one hardware processor to perform the method of any one of Examples D1-D16.

E1. A method, comprising:

obtaining data regarding light detected over time from luminescent labels associated with at least one molecule undergoing a sequencing reaction;

evaluating a first candidate change point of the data as being a change point;

evaluating, for a portion of the data occurring at a time after the first candidate change point, a second candidate change point as being a change point; and updating a set of change points to include the second candidate change point.

E2. The method of Example E1, wherein evaluating the second candidate change point further comprises evaluating candidate change points occurring at times after the first candidate change point.
E3. The method of Example E1, wherein evaluating the first candidate change point is performed at least in part by using at least one statistical model having a parameter representing the likelihood of having a change point at a particular time.
E4. The method of Example E3, wherein evaluating the first candidate change point further comprises using the at least one statistical model to calculate a score for the first candidate change point.
E5. The method of Example E4, wherein evaluating the first candidate change point further comprises calculating a value of the parameter for the first candidate change point and calculating the score based on the value of the parameter.
E6. The method of Example E3, wherein evaluating the first candidate change point further comprises calculating values of the parameter associated with different data points within the data and determining the first candidate change point is a change point based on the values.
E7. The method of Example E3, wherein evaluating the second candidate change point is performed at least in part by using the at least one statistical model.
E8. The method of Example E7, wherein evaluating the second candidate change point further comprises using the at least one statistical model to calculate a score for the second candidate change point.
E9. The method of Example E8, wherein evaluating the second candidate change point further comprises calculating a value of the parameter for the second candidate change point and calculating the score based on the value of the parameter.
E6. The method of Example E7, wherein evaluating the second candidate change point further comprises calculating values of the parameter associated with different times occurring after the first candidate change point and determining the second candidate change point is a change point based on the values.
E7. The method of Example E1, wherein the method further comprises determining at least one nucleotide incorporation event based on the set of change points.
E8. The method of Example E1, wherein the method further comprises determining segments of the data between individual change points of the set of change points as being nucleotide incorporation events.
E9. The method of Example E1, wherein the method further comprises determining segments of the data between individual change points of the set of change points as being background signal.
E10. The method of Example E1, wherein obtaining the data occurs during the sequencing reaction.
E11. A system comprising:

at least one hardware processor; and

at least one non-transitory computer-readable storage medium storing processor-executable instructions that, when executed by the at least one hardware processor, cause the at least one hardware processor to perform the method of any one of Examples E1-E10.

E12. At least one non-transitory computer-readable storage medium storing processor-executable instructions that, when executed by at least one hardware processor, cause the at least one hardware processor to perform the method of any one of Examples E1-E10.
E13. A sequencing instrument, comprising:

at least one photodetector configured to receive light from luminescent labels during a sequencing reaction;

at least one hardware processor; and

at least one non-transitory computer-readable storage medium storing processor-executable instructions that, when executed by the at least one hardware processor, cause the at least one hardware processor to perform the method of any one of Examples E1-E10.

The terms “program” or “software” are used herein in a generic sense to refer to any type of computer code or set of processor-executable instructions that can be employed to program a computer or other processor to implement various aspects of embodiments as discussed above. Additionally, it should be appreciated that according to one aspect, one or more computer programs that when executed perform methods of the disclosure provided herein need not reside on a single computer or processor, but may be distributed in a modular fashion among different computers or processors to implement various aspects of the disclosure provided herein.

Processor-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in one or more non-transitory computer-readable storage media in any suitable form. For simplicity of illustration, data structures may be shown to have fields that are related through location in the data structure. Such relationships may likewise be achieved by assigning storage for the fields with locations in a non-transitory computer-readable medium that convey relationship between the fields. However, any suitable mechanism may be used to establish relationships among information in fields of a data structure, including through the use of pointers, tags or other mechanisms that establish relationships among data elements.

Also, various inventive concepts may be embodied as one or more processes, of which examples have been provided. The acts performed as part of each process may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, and/or ordinary meanings of the defined terms.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the techniques described herein in detail, various modifications, and improvements will readily occur to those skilled in the art. Such modifications and improvements are intended to be within the spirit and scope of the disclosure. Accordingly, the foregoing description is by way of example only, and is not intended as limiting. The techniques are limited only as defined by the following claims and the equivalents thereto.

What is claimed is:

1. A method, comprising:
obtaining data regarding light detected over time from luminescent labels associated with at least one molecule undergoing a sequencing reaction;
evaluating candidate change points of the data within a time window of the data that varies over time; and
outputting information identifying a set of change points based on evaluating the candidate change points.

2. The method of claim 1, wherein obtaining the data further comprises receiving portions of the data at different times.

3. The method of claim 2, wherein the method further comprises, after receiving at least one portion of the data, adjusting the time window of the data to include the at least one portion of data.

4. The method of claim 1, wherein evaluating the candidate change points further comprises estimating a change point of the data and evaluating candidate change points of the data occurring after the change point.

5. The method of claim 4, wherein evaluating candidate change points of the data occurring after the change point further comprises using at least a portion of data occurring prior to the change point.

6. The method of claim 1, wherein evaluating the candidate change points is performed at least in part by:
evaluating candidate change points of the data within a first time window of the data;
generating a second time window at least in part by adding, to the first time window, at least one data point occurring after the first time window; and
evaluating candidate change points of the data within the second time window.

7. The method of claim 1, wherein evaluating the candidate change points is performed at least in part by:
estimating a first change point of the data by evaluating a first set of candidate change points; and
estimating a second change point of the data by evaluating a second set of candidate change points occurring after the first change point.

8. The method of claim 1, wherein evaluating the candidate change points is performed at least in part by using at least one statistical model that estimates the likelihood of the candidate change points being change points.

9. A method, comprising:
obtaining data regarding light detected over time from luminescent labels associated with at least one molecule undergoing a sequencing reaction; and
detecting change points of the data during the sequencing reaction.

10. The method of claim 9, wherein the method further comprises determining at least one nucleotide incorporation event at least in part based on the detected change points.

11. The method of claim 10, wherein the method further comprises:
assigning the at least one nucleotide incorporation event to different types of nucleotides;
generating, based on the assigning, a nucleotide sequence; and
outputting information identifying the nucleotide sequence.

12. The method of claim 9, wherein detecting change points further comprises evaluating candidate change points of the data during the sequencing reaction.

13. The method of claim 9, wherein the method further comprises determining at least one nucleotide incorporation event based on the detected change points.

14. The method of claim 9, wherein the method further comprises determining segments of the data between individual change points as corresponding to background signal.

15. The method of claim 14, wherein determining the segments of the data further comprises comparing values of at least one feature of the data within the segments to a threshold value, and identifying segments having values of the at least one feature below the threshold value as corresponding to background signal.

16. A method, comprising:
obtaining first data regarding light detected from at least one luminescent label associated with at least one molecule undergoing a sequencing reaction;
categorizing at least one first candidate change point of the first data as not being a change point;
after the categorizing, obtaining second data regarding light detected from at least one luminescent label associated with the at least one molecule undergoing the sequencing reaction; and
evaluating at least one second candidate change point based at least in part on the second data.

17. The method of claim 16, wherein categorizing the at least one first candidate change point further comprises determining that a change point does not exist within the first data.

18. The method of claim 16, wherein categorizing the at least one first candidate change point is performed at least in part by using at least one statistical model having a parameter representing a likelihood of having a change point at a particular time.

19. The method of claim 18, wherein categorizing the at least one first candidate change point further comprises calculating at least one value of the parameter associated with the at least one first candidate change point and using the at least one value to determine that the at least one first candidate change point is not a change point.

20. The method of claim 19, wherein categorizing the at least one first candidate change point further comprises comparing the at least one value to a threshold value.
